(19) United States
(12) Patent Application Publication
(10) Pub. No.: US 2023/0176038 A1
(43) Pub. Date: Jun. 8, 2023

(54) Methods for Identifying Modulators of Insect Transient Receptor Potential Channels

(71) Applicant: BASF Corporation, Florham Park, NJ (US)

(72) Inventors: Alexandre NESTEROV, Chapel Hill, NC (US); Ramani KANDASAMY, Chapel Hill, NC (US); Lynn F. STAM, Raleigh, NC (US)

(21) Appl. No.: 17/787,968

(22) PCT Filed: Dec. 10, 2020
(86) PCT No.: PCT/US2020/064296

§ 371 Date: Jun. 22, 2022

Related U.S. Application Data
(60) Provisional application No. 62/952,756, filed on Dec. 23, 2019.

(51) Int. Cl. G01N 33/50

(52) U.S. Cl. CPC G01N 33/5041; G01N 2333/43552

(57) ABSTRACT

The present invention relates to a screening method for determining whether or not a candidate compound is a modulator of a complex consisting of the insect transient receptor potential channel Nanchung protein and the insect transient receptor potential channel Water witch protein. The present invention further relates to a screening method for determining whether or not a candidate compound directly binds to the complex consisting of the Nanchung protein and Water witch protein. The present invention further relates to a screening method for determining whether or not a candidate compound is a modulator of the insect Water witch protein. The present invention further provides a method of insect control by applying an insect-specific TRP channel modulator determined by the screening method. The present invention further provides a method of classifying an insecticide compound by its mode of action using the binding assay and/or a functional assay.

### CROSS-REFERENCE TO RELATED APPLICATION

This application claims the benefit of U.S. Provisional Pat. Application No. 62/952,756, filed Dec. 23, 2019, which is incorporated by reference herein in its entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is “SEQ LISTING 180852WO01.” The size of the text file is 702 KB, and the text file was created on Dec. 8, 2020.

### BACKGROUND

The present invention relates to compositions and methods of insect control. The present invention generally relates to methods for determining agents that directly bind to and/or modulate a biological activity of the insect transient receptor potential A (TRPA) Water witch protein and/or complexes consisting of the insect transient receptor potential V (TRPV) Nanchung protein and the insect transient receptor potential A (TRPA) Water witch protein. The present invention also relates to compositions and methods of mode of action classification for an insecticide composition comprising a compound capable of modulating biological activity of the insect transient receptor potential A (TRPA) Water witch protein and/or complexes consisting of the insect transient receptor potential V (TRPV) Nanchung protein and the insect transient receptor potential A (TRPA) Water witch protein in a cell. The present invention further relates to compositions and methods of mode of action classification for an insecticide composition comprising a compound capable of directly binding complexes consisting of the insect transient receptor potential V (TRPV) Nanchung protein and the insect transient receptor potential A (TRPA) Water witch protein.

Almost all field crops, plants, and commercial farming areas are susceptible to attacks by plant insect pests. Plant insect pests are a major factor in the loss of the world’s commercially important agricultural crops resulting both, in economic hardship to farmers and nutritional deprivation for local populations in many parts of the world. In other words, insect pests cause great loses and damages to human agriculture, food supply, post-harvest storage, horticulture, animal health and public health.

While advances have been made in the control of these insects, these insects have been able to adopt and evade the control measures, resulting in resistant insect populations (Perry et al., 2011). As a result, there remains a need for better understanding of the mechanisms that underlie feeding and other behaviors of insect pests. Such knowledge would allow for the design of agents and strategies for intervening or preventing attacks on field crops, plants, and commercial farming areas by the insect pests or prevent spreading of human and animal diseases.

Broad spectrum chemical pesticides and insecticides have been used extensively to control or eradicate insect pests. There is, however, substantial interest in identifying and/or developing effective alternative insecticides. As such, effective methods for screening of the new insecticides would be desirable.

Several species or arthropods, including mosquitos, flies, ticks, midges and flees transmit variety of pathogenic viruses and parasites. Widely used insect repellents such as DEET, picaridin and permethrin have raised several concerns in terms of environmental and health risks (Lee, 2018). There is, therefore, substantial interest in developing new methods to screen for efficient repellents with potentially low toxisity to health and environment.

What are needed, then, are new methods that can be employed in screening for agents capable of binding to and/or modulating biological activity of the insect transient receptor potential A (TRPA) Water witch protein and/or complexes consisting of the insect transient receptor potential V (TRPV) Nanchung protein and the insect transient receptor potential A (TRPA) Water witch protein, which, in some cases can modulate insect behavior and act as insecticides and/or insect repellents.

### SUMMARY

The present invention relates to a method for determining the mode of action classification for the candidate compound according to the Insecticide Resistance Action Committee (IRAC) using one or more of the following: functional assay and/or binding assay.

For example, in certain embodiments, a functional assay may be used to determine whether a candidate compound acts as a modulator of the insect transient receptor potential A (TRPA) Water witch protein and/or complexes consisting of the insect transient receptor potential V (TRPV) Nanchung protein and the insect transient receptor potential A (TRPA) Water witch protein in a cell.

In yet another embodiment, a functional assay may be used to determine whether or not a candidate compound acts in a similar way as endogenous modulator of complexes consisting of the insect transient receptor potential V (TRPV) Nanchung protein and the insect transient receptor potential V (TRPV) Inactive protein in a cell.

In yet another embodiment, a functional assay may be used to determine whether or not a candidate compound acts in a similar way as endogenous modulator of complexes consisting of the insect transient receptor potential V (TRPV) Nanchung protein and the insect transient receptor potential A (TRPA) Water witch protein in a cell.

The functional assay may include at least one of the following steps: (1) detecting calcium ion mobilization in the first cell in response to the candidate compound or endogenous modulator, (2) detecting a membrane potential in the first cell in response to the candidate compound on endogenous modulator, (3) comparing calcium ion mobilization in the first cell in the absence of the candidate compound or endogenous modulator with calcium ion mobilization in the first cell in the presence of the candidate compound or endogenous modulator, (4) comparing a membrane potential in the first cell in the absence of the candidate compound or endogenous modulator with a membrane potential in the first cell in the presence of the candidate compound or endogenous modulator, (5) comparing calcium ion mobilization in the first cell in the presence of the candidate compound or endogenous modulator with calcium ion mobilization reference level indicative of no modulation of the TPRV channel, and/or TRPA channel, and/or complexes consisting of the insect transient receptor potential V (TRPV) and the insect transient receptor potential A (TRPA) channel (6) comparing a membrane potential in the first cell in the presence of the candidate compound or endogenous modulator with a membrane potential reference level indicative of no modulation of the TPRV channel and or TRPA channel and/or complexes consisting of the insect transient receptor potential V (TRPV) channel and the insect transient receptor potential A (TRPA) channel. Preferably, the candidate compound or endogenous modulator may modulate the calcium ion mobilization or the membrane potential in the first cell by at least 20% relative to the reference level. The candidate compound or endogenous modulator may be a modulator that inhibits the activities of the insect TRPV channel and/or the insect TRPA channel and/or complexes consisting of the insect transient receptor potential V (TRPV) channel and the insect transient receptor potential A (TRPA) channel. Alternatively, the candidate compound or endogenous modulator may be a modulator that activates the insect TRPV channel and or TRPA channel and/or complexes consisting of the insect transient receptor potential V (TRPV) channel and the insect transient receptor potential A (TRPA) channel. The candidate compound may be a modulator that kills insects and/or inhibits insect feeding and/or perturbs insect behaviour.

In another embodiment a bininding assay may be used to determine if the candidate compound binds directly to complexes consisting of transient receptor potential (TRPV) proteins and transient receptor potential A (TRPA) proteins.

In another embodiment a binding assay can be used to determine whether or not a candidate compound binds to the same site as endogenous modulator of complexes consisting of the insect transient receptor potential V (TRPV) proteins and the insect transient receptor potential A (TRPA) proteins.

In another embodiment a binding assay can be used to determine whether or not a candidate compound binds to the same site as endogenous modulator of complexes or channels consisting of transient receptor potential (TRPV) proteins and transient receptor potential A (TRPA) proteins.

In another embodiment a binding assay can be used to determine whether or not a two or more candidate compounds binds to the same or different sites on complexes formed by the transient receptor potential (TRPV) proteins and transient receptor potential A (TRPA) proteins.

A binding assay may include (1) providing one or more Nanchung proteins, transient receptor potential V (TRPV) Inactive proteins, and/or transient receptor potential A (TRPA) Water witch proteins which may be located in a cell, derived from a cell, produced by a cell or produced in a cell-free system; (2) contacting the one or more TRPV Nanchung proteins, and/or TRPV Inactive proteins, and/or TRPA Water witch proteins and/or complexes consisting of the insect transient receptor potential V (TRPV) Nanchung protein and the insect transient receptor potential A (TRPA) Water witch protein wherein the binding affinity of the candidate compound is indicative of the candidate compound’s ability to directly bind to one or more Nanchung proteins, TRPV Inactive proteins and/or TRPA Water witch proteins, and/or complexes consisting of the insect transient receptor potential V (TRPV) Nanchung protein and the insect transient receptor potential A (TRPA) Water witch protein.

For example, in certain embodiments, the binding assay step may include at least one of the following steps: (1) contacting the one or more TRPV Nanchung proteins and/or TRPV Inactive proteins and/or TRPA Water witch proteins and/or complexes consisting of TRPV Nanchung protein and TRPA Water witch protein with a compound, wherein the compound may be labeled or unlabeled; (2) measuring the amount of the compound which is bound to the one or more Nanchung proteins, TRPV Inactive proteins and/or TRPA Water witch proteins, and/or complexes consisting of TRPV Nanchung protein and TRPA Water witch protein protein; (3) calculating the equilibrium dissociation constant (K_{d}) for the one or more compounds; (4) wherein the K_{d} may be determined using one or more of the following methods: (I)(a) maximum binding (Bₘₐₓ) and (b) the concentration of labeled compound needed to reach 50% of maximum binding, (II) fluorescence polarization assay, (III) surface plasmon resonance, (IV) isothermal calorimetry, (V) Schild analysis; (5) contacting the one or more Nanchung proteins, and/or TRPV Inactive proteins and/or TRPA Water witch proteins and/or complexes consisting of TRPV Nanchung protein and TRPA Water witch protein with a labeled compound having high affinity to one or more Nanchung proteins, and/or TRPV Inactive proteins and/or TRPA Water witch proteins and/or complexes consisting of TRPV Nanchung protein and TRPA Water witch protein; (6) contacting the one or more Nanchung proteins, TRPV Inactive proteins and/or TRPA Water witch proteins, one or more Nanchung proteins, and/or TRPV Inactive proteins and/or TRPA Water witch proteins and/or complexes consisting of TRPV Nanchung protein and TRPA Water witch protein with a candidate compound or endogenous modulator in the presence of the labeled compound of step 1; (7) increasing the amount of the candidate compound or endogenous modulator available to contact the one or more Nanchung proteins, TRPV Inactive proteins and/or TRPA Water witch proteins, one or more Nanchung proteins, and/or TRPV Inactive proteins and/or TRPA Water witch proteins and/or complexes consisting of TRPV Nanchung protein and TRPA Water witch protein until binding of the labeled compound is near 0%; (8) calculating the equilibrium inhibition constant (Ki) and/or the equilibrium dissociation constant (K_{d}) for competition assay using one or more of the following methods: (I) (a) determining amount of bound labeled compound in the absence of the candidate compound or endogenous modulator and (b) determining concentration of the candidate compound or endogenous modulator that is required to reduce the amount of bound labeled compound by 50%, (II) fluorescent polarization assay, and/or (III) Schild analysis; (9) comparing the inhibition constant of the candidate compounds or endogenous modulator to other candidate compounds; (10) comparing the inhibition constant of the candidate compounds or endogenous modulator to the dissociation constant of the labeled compound; (11) comparing the inhibition constant of the candidate compound to the inhibition constant of the one or more candidate compounds.

In the methods set forth herein, the one or more insect TRPV channels and/or the one or more insect TRPA channels may comprise one or more TRPV proteins and/or one or more TRPA proteins in a complex. In the methods set forth herein, the one or more insect TRPV channels and/or TRPA channels may comprise one or more Nanchung proteins, Inactive proteins, Water witch proteins, and/or Nanchung and Inactive protein complexes, and/or Nanchung and Water witch protein complexes. For clarity, the TRPV and/or TRPA channels may also comprise a Nanchung protein and/or Inactive proteins and/or Water witch one or more Nanchung proteins, and/or TRPV Inactive proteins and/or TRPA Water witch proteins and/or complexes consisting of TRPV Nanchung protein and TRPA Water witch proteins found in cellular membranes, inserted in artificial membranes, inserted in Nanchungodisks, solubilized in detergents, solubilized in non-detergent polymers, solubilized in amphipols and/or produced in a cell-free system. It is to be understood that the Nanchung, Inactive, and Water witch, proteins and/or one or more Nanchung proteins, and/or TRPV Inactive proteins and/or TRPA Water witch proteins and/or complexes consisting of TRPV Nanchung protein and TRPA Water witch protein may be found in cellular membranes, in solution, or in a cell-free system. It is to be understood that the Nanchung protein , Inactive protein, Water witch protein, and/or complexes consisting of TRPV Nanchung protein and TRPA Water witch protein may be contained within one or more cellular membranes, one or more pieces of cellular membranes, produced in a cellular system, produced in a cell-free system, and/or from cultured cells in a petri dish, or from cells or tissues derived from experimental animals or experimental insects. For purposes of determining whether a candidate compound directly binds to the insect TRPV proteins and/or TRPA proteins the TRPV and/or TRPA channels may comprise one or more Nanchung, Inactive or Water witch proteins.

It is to be understood that Nanchung and Inactive proteins are both insect TRPV proteins. Water witch is insect TRPA protein. Nanchung proteins may form a complex with Inactive proteins and/or Water witch proteins. The candidate compound may be a small organic molecule, small inorganic molecule, polysaccharides, peptides, proteins, nucleic acids, an extract made from biological materials, and any combination thereof. The endogenous modulator may be a small organic molecule, small inorganic molecule, polysaccharides, peptides, proteins, nucleic acids, an extract made from biological materials, and any combination thereof.

In another embodiment, the methods set forth above may use one or more of the following compounds as the labeled compound: afidopyropen, pymetrozine, and/or N-deacetylated form of pyrifluquinazon.

In yet another embodiment, the present invention relates to a candidate compound selected by the methods described above. The candidate compound may be a modulator that inhibits or increases the activity of the insect TRPV channel or insect TRPA channel. The candidate compound may be a modulator that acts as repellent and/or inhibits a feeding behavior of an insect. Even further, the candidate compound may modulate and/or bind directly to the one or more TRPV Nanchung proteins and/or, TRPV Inactive proteins and/or TRPA proteins, and/or complexes consisting of TRPV Nanchung proteins and TRPV Inactive proteins and/or complexes consisting of TRPV Nanchung proteins and TRPA Water witch proteins. The candidate compound may further modulate and/or bind directly to one or more insect Nanchung TRPV proteins, and/or insect TRPV Inactive proteins and/or insect TRPA Water witch proteins, and/or complexes consisting of TRPV Nanchung proteins and TRPV Inactive proteins and/or complexes consisting of TRPV Nanchung proteins and TRPA Water witch proteins and in particular to the same site on insect Nanchung TRPV proteins, and/or insect TRPV Inactive proteins and/or insect TRPA Water witch proteins, and/or complexes consisting of TRPV Nanchung proteins and TRPV Inactive proteins and/or complexes consisting of TRPV Nanchung proteins and TRPA Water witch proteins. The candidate compound may further modulate and/or bind directly to one or more arthropod Nanchung TRPV proteins, and/or arthropod TRPV Inactive proteins and/or arthropod TRPA Water witch proteins, and/or complexes consisting of arthropod TRPV Nanchung proteins and arthropod TRPV Inactive proteins and/or complexes consisting of arthropod TRPV Nanchung proteins and arthropod TRPA Water witch proteins. The candidate compound may further modulate and/or bind directly to one or more nematode Nanchung TRPV proteins, and/or nematode TRPV Inactive proteins and/or nematode TRPA Water witch proteins, and/or complexes consisting of nematode TRPV Nanchung proteins and nematode TRPV Inactive proteins and/or complexes consisting of nematode TRPV Nanchung proteins and nematode TRPA Water witch proteins and/or other non-target/non-pest species.

In another embodiment, the present invention relates to a composition containing the candidate compound identified by one or more of the methods set forth herein.

In another embodiment, the present invention relates to a method of insect control that includes applying to an insect or to an insects habitat, feeding source, and/or environment/habitat a composition comprising the candidate compound selected by one or more of the methods described herein or a compound selected by one or more of the methods described above. The compound may be an inhibitor of an insect TRPV and/or TRPA channel. The compound may be an activator of an insect TRPV and or TRPA channel. The compound may bind directly to the insect Nanchung protein, and or Water witch protein. The insect may be an agricultural/horticultural pest or a disease vector or a parasite.

### BRIEF DESCRIPTION OF THE DRAWINGS

FIG. 1 depicts a schematic illustration of the expression cassette for insect TRP channels in an adenovirus expression vector.

FIGS. 2A and 2B depict dose response relationships of calcium responses evoked by afidopyropen (AP), and pymetrozine (PM), but not Flonicamid (FL) or TFNA-AM in cells co-expressing Water witch and Nanchung (Wtrw + Nan), but not in cells expressing Water witch alone (Wtrw).

FIG. 3 depicts physical association between Nanchung (Nan-HA) and Water witch (Wtrw-FLAG) proteins in co-immunoprecipitation experiment.

FIG. 4A depicts activation of Ca²⁺ mobilization as a function of time by endogenous agonist, nicotinamide in mammalian cells co-expressing Nanchung and Inactive (Nan + Iav), or in mammalian cells co-expressing Nanchung and Water witch (Nan + Wtrw) but not in parental mammalian cells (cells are cells prior to infection with the genes or proteins of interest), nor in cells co-expressing Inactive and Water witch (Iav + Wtrw), no in cells expressing Nanchung (Nan), Inactive (Iav) or Water witch (Wtrw) individually. FIG. 4B depicts dose response relationships of calcium responses evoked by nicotinamide in cells co-expressing Nanchung and Inactive (Nan + Iav) or cells co-expressing Nanchung and Water witch (Nan + Wtrw).

FIG. 5A depicts [3H]-afidopyropen saturation binding to Nanchung (Nan) protein alone, Water witch (Wtrw) protein alone and complexes formed by Nanchung protein and Water witch protein (Nan + Wtrw). Binding affinities (K_{d}) are shown in parenthesis. FIG. 5B depicts Scatchard transformation of the saturation binding and increased binding affinity of complexes formed by Nanchung protein and Water witch protein (Nan + Wtrw), as compared to Nanchung protein individually (Nan).

FIG. 6A depicts competition between [³H]-afidopyropen and unlabeled pymetrozine for binding to complexes formed by Nunchung protein and Inactive protein or complexes formed by Nanchung protein and Water witch protein. FIG. 6B depicts competition between [³H]-afidopyropen and unlabeled nicotinamide for binding to complexes formed by Nunchung protein and Inactive protein or complexes formed by Nanchung protein and Water witch protein.

FIGS. 7A and 7B depict activation of Ca2+ mobilization by plant-derived deterrents cinnamaldehyde (A), and allyl isothiocyanate (B) in mammalian cells expressing Water witch protein (Wtrw), in cells co-expressing Nanchung and Water witch proteins (Nan + Wtrw) , and in cells co-expressing Inactive and Water witch, but not in parental mammalian cells, nor in cells expressing Nancung (Nan), Inactive (Iav), or cells co-expressing Nanchung and Inactive proteins (Nan + Iav).

FIG. 8 depicts amino acid alignment between homologous regions of Inactive (Iav) and Water witch (Wtrw) proteins and shows limited (22%) identity. Horisontal bars indicate positions of predicted transmembrane domains.

FIGS. 9A through 9K depict sequence alignments of the Nanchung proteins (SEQ ID NOS 64-90), all respectively, in order of appearance.

FIGS. 10A through 10J depict sequence alignments of the Water witch proteins (SEQ ID NOS 91-126), all respectively, in order of appearance.

FIG. 11 depicts an alignment tree for Nanchung proteins.

FIG. 12 depicts an alignment tree for Water witch proteins.

### DETAILED DESCRIPTION OF THE DRAWINGS AND THE PRESENTLY PREFERRED EMBODIMENTS

### I. General Considerations

The transient receptor potential (TRP) channels constitute a large and important class of channels involved in modulating cellular homeostasis (Wu et al., 2010). The present invention provides methods and compositions that identify or include one or more compounds or endogenous modulators that bind to and/or modulate at least one TRP family member, including, but not limited to TRPV and TRPA channels in an insect.

Specifically, the present invention provides methods for identifying candidate compounds or endogenous modulators capable of directly binding and modulating protein complexes consisting of TRPV protein Nanchung and TRPA protein Water witch.

The present invention further provides methods for identifying candidate compounds capable of directly modulating TRPA protein Water witch.

Modulating an insect TRPV and/or TRPA channels may modulate calcium homeostasis, sodium homeostasis, intracellular calcium levels, membrane polarization (resting membrane potential), and/or cation levels in a cell. Compounds that can modulate one or more insect TRPV and/or insect TRPA proteins and/or complexes formed by the insect TRPV proteins and TRPA proteins are useful in many aspects including, but not limited to, insect control. Knowing wether or not a compound directly binds or modulates TRPV proteins and/or TRPA proteins and/or complexes formed by the insect TRPV proteins and TRPA proteins and knowing wheter or not compounds bind to the same or different sites of an insect TRPV protein and/or insect TRPA protein and/or complexes formed by the insect TRPV proteins and TRPA proteins is critical for insect management planning and preventing the development of resistant insect populations.

The present invention identifies afidopyropen, pymetrozine and endogenous compound nicotinamide modulating biological activity of complexes formed by the insect TRPV Nanchung protein and insect TRPA Water witch protein for purposes of the determination of the biological activity by the calcium mobilization assay set herein.

The present invention further identifies cinnamaldehyde, and allyl isothiocyanate modulating biological activity of TRPA Water witch protein and/or complexes formed by the insect TRPV Nanchung protein and insect TRPA Water witch protein for purposes of the determination of the biological activity by the calcium mobilization assay set herein.

The present invention further identifies afidopyropen having affinity to complexes or channels consisting of TRPV Nanchung protein and TRPA Water witch protein such that one or more of the compounds above may be used in the methods presented herein as a labeled compound for purposes of the determination of the binding affinity of a labeled compound by saturation binding assay set forth herein.

The present invention further identifies afidopyropen, pymetrozine and endogenous modulator nicotinamide as compounds having affinity to complexes or channels consisting of TRPV Nanchung protein and TRPA Water witch such that one or more of the compounds above may be used as a labeled compound and another one used as unlabeled compound for purposes of the determination of binding affinity of unlabeled compound by competition binding assay set forth herein.

The present invention also provides compositions comprising the candidate compounds identified herein for use as insecticides and/or insect repellents for treating pests, plants and crops. The present invention further comprises classifying the candidate compounds identified herein by their mode of action.

Without being bound by theory, the modulation of an insect TRPV proteins, and/or TRPA proteins and/or complexes formed by the insect TRPV proteins and insect TRPA proteins elicits signaling pathway that brings forth sensory neuron modulation which may kill insects, alter insect behavior or repel insects. Thus, again without being bound by theory, it is believed that compounds that modulate (e.g., activate) an insect TRPV proteins and/or TRPA proteins and/or complexes formed by the insect TRPV proteins and insect TRPA proteins may be used as insecticides or insect repellents.

Transient receptor potential (TRP) proteins form cation channels, which have been classified into at least seven groups: TRPC (canonical), TRPV (vanilloid), TRPM (melastatin), TRPP (polycystins), TRPML (mucolipins), TRPA (ankyrin), and TRPN (no mechanosensor potential C), which is not found in mammals (Fowler and Montell, 2013).

All TRP channels exist as tetramers, which can function either as homotertamers or form heterotetramers with other members of the same or different subfamilies. TRP channels can be modulated by a variety of physical and chemical stimuli and are involved in virtually every sensory modality (Fowler and Montell, 2013).

The TRPV channels are present in a great number of organisms, including, e.g., insects (Drosophila, Tribolium, Pediculus, Culex, and Anopheles), crustaceans (Daphnia pulex) nematodes (Caenorhabditis elegans), molluscs (Aplysia californica, Lottia gigantea) and mammals (humans, mice, rats, monkeys and chimpanzee).

Currently, the mammalian TRPV family has 6 members: TRPV1, TRPV2, TRPV3, TRPV4, TRPV5 and TRPV6. Mammalian TRPVs are involved in a broad array of sensory functions, including pain perception, temperature, osmolarity, touch, and taste (Venkatachalam and Montell, 2007). In addition to physical stimuli, mammalian TRPVs can be either activated or inhibited by varity of endogenous and exogenous chemical compounds (Vriens et al., 2009).

Nematodes have 5 members of TRPV family: OCR-1, OCR-2, OCR-3, OCR-4 and OSM-9. Nematode TRPVs are involved in various sensory functions, including osmolarity, olfaction, and mecanosensation (Upadhyay et al., 2016).

Arthropods, and more particularly insects have two members of TRPV family: Nanchung (Nanchung), and Inactive (Inactive) which are most closely related to nematode OCR-4 and OSM-9 respectfully (Matsuura et al., 2009). Nanchung and Inactive can form tightly associated heteromers. Nanchung and Inactive heteromers are formed exclusively in mechanosensory organs known as chordotonal organs (Nesterov et al., 2015).

Chordotonal organs are unique to arthropods, are located in insect joints and are activated by stretch resulted from relative rotation of insect body segments (Kavlie and Albert, 2013). Activation of Nanchung and Inactive heteromers by stretch generates nerve signal, which transduces information about gravity, sound, and relative positions of body parts (Gong et al., 2004; Kim et al., 2003; Nesterov et al., 2015).

At least three commercial insecticides, afidopyropen, pymetrozine and pyrifluquinazon overstimulate and eventually silence Nanchung and Inactive heteromers, perturbing insect coordination and inhibiting feeding, which eventually leads to death by dessication and starvation (Nesterov et al., 2015).

Afidopyropen, pymetrozine and pyrifluquinazon can directly bind Nanchung protein alone, but binding affinity is greatly increased by formation of Nanchung and Inactive heteromers (Kandasamy et al., 2017). Formation of Nanchung and Inactive heteromers is essential for biological activity and insecticide action, since neither Nanchung nor Inactive alone can be activated by any of the three insecticides (Kandasamy et al., 2017; Nesterov et al., 2015).

Pymetrozine may also act as insect repellent, although the molecular mechanism of repulsion remains obscure (Chang and Snyder, 2008).

In addition to the insecticides, both insect Nanchung and Inactive, as well as their nematode orthologs, OCR-4 and OSM-9 can be activated by, nicotinamide, a form of vitamin B3 (Upadhyay et al., 2016). Nicotinamide can be both absorbed from diet and produced engoneously. Analogous to activation of Nanchung and Inactive by the insecticides activation by nicotinamide required co-expression of either insect Nanchung plus Inactive or nematode OCR-4 plus OSM-9.

Although the effects of either insecticides or nicotinamide requires simultaneous presence of Nanchung and Inactive, it has not ben shown wheter or not aforementioned insecticides and nicotinamide bind to the same or different sites in Nanchung and Inactive heteromers.

In chordotonal organs Nanchung and Inactive are interdependent: loss of Inactive abolishes expression of Nanchung and vice versa (Gong et al., 2004; Nesterov et al., 2015), suggesting that in chordotonal organs formation of Nanchung and Inactive heteromers is essential for stability of both Nanchung and Inactive proteins. In some other insect tissues, however. Nanchung and Inactive can function independently of each other. In particular, Nanchung, but not Inactive is required to detect food hardness by fruit fly labellar mechanosensory neurons (Jeong et al., 2016). Likewise, Nanchung, but not Inactive is required for defensive kicking behavior in fruit fly wing margin bristles (Li et al., 2016). These observations raise a possibility that in certain tissues Nanchung may partner with some other TRP channel to for protein stability and functional response.

Commercial insecticide flonicamid (Maienfisch, 2012) as well as its more active derivative, 4-trifluoronicotinamide (TFNA-AM) (Salgado et al., 2014) act on insect chordotonal organs and alter insect feeding behavior in a manner similar to afidopyropen, pymetrozine and purifluquinazon. These compounds, however, do not directly bind or modulate insect TRPV channels and are thought to have different, still unknown molecular target (Kandasamy et al., 2017).

There is one identified TRPA gene in mammals (Wu et al., 2010) and six TRPA genes in insects (Matsuura et al., 2009). Different members of TRPA family are present in different insect species. Fruit flies have four TRPA channels: TRPA1, Painless, Pyrexia, and Water witch.

Insect TRPA1 channels are involved in sensation of temperature, mechanical stimulation and light (Fowler and Montell, 2013).

Insect TRPA1 channels can be activated by several plant-derived insecticides and/or repellents including cinnamaldehyde and allyl isothiocyanate (Kang et al., 2010).

Cinamaldehyde and allyl isothiocyanate modulate TRPA1 channels by covalently modifying specific cysteine and lysine amino acids in TRPA1 protein (Hinman et al., 2006; Macpherson et al., 2007).

Painless is involved in sensation of temperature, mechanical stimulation and gravity (Fowler and Montell, 2013).

Pyrexia is involved in sensation of temperature and gravity (Fowler and Montell, 2013).

Water witch is involved in distinguishing moist from dry air in fruit flies (Liu et al., 2007), and recovery from tonic immobility in beetles (Kim et al., 2015). In fruit flies Water witch is expressed in hydroreceptors (Liu et al., 2007) and in chordotonal organ of the second segment of antennae (Senthilan et al., 2012). Replacement of the conserved glutamic acid to lysine (E754K) in fruit fly’s Water witch (Cooper et al., 2008), a point mutation, which abolishes ion conductance by other TRP channels (Li et al., 2007) impairs fruit flies sound perception (Senthilan et al., 2012).

Modulation of fruit fly TRPA-1 channel is thought to be the predominant mode of action of plant-derived repellents cinnamaldehyde and allyl isothiocyanate (Kang et al., 2010).

Based on amino acid composition Painless, Pyrexia and Water witch were predicted to be not responsive to cinnamaldehyde and allyl isothiocyanate (Kang et al., 2010).

Cinnamaldehyde and allyl isothiocyanate failed to directly modulate Painless (Sokabe et al., 2008)

Thus far, there are no reported chemichal modulators of Water witch.

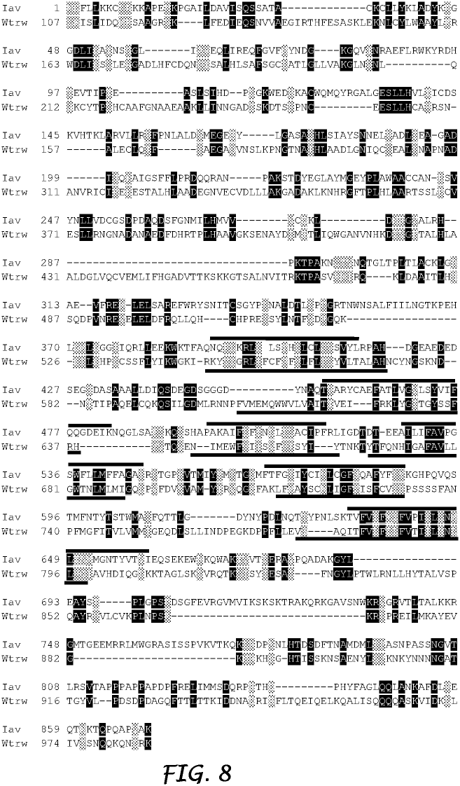

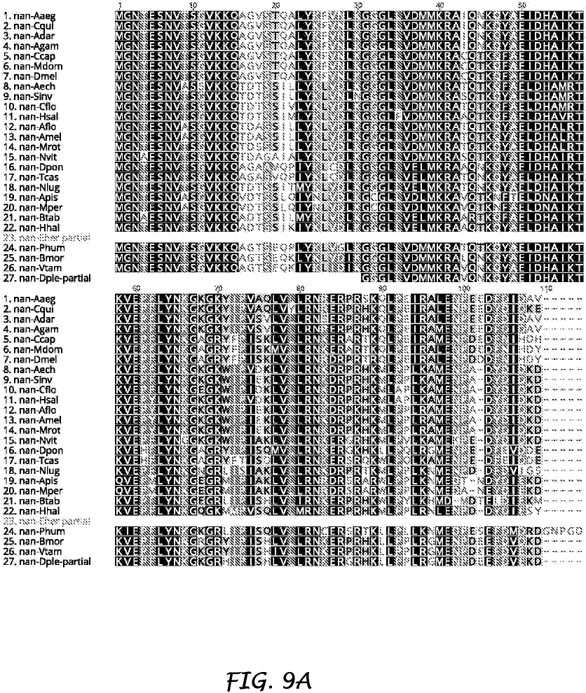

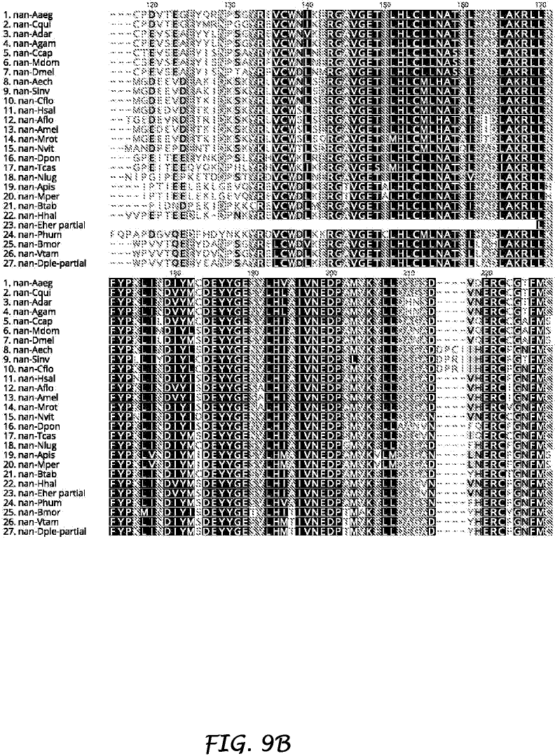

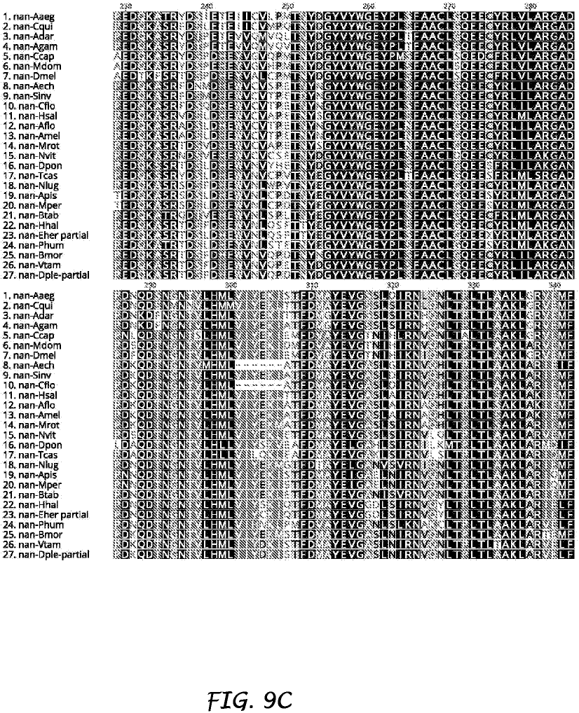

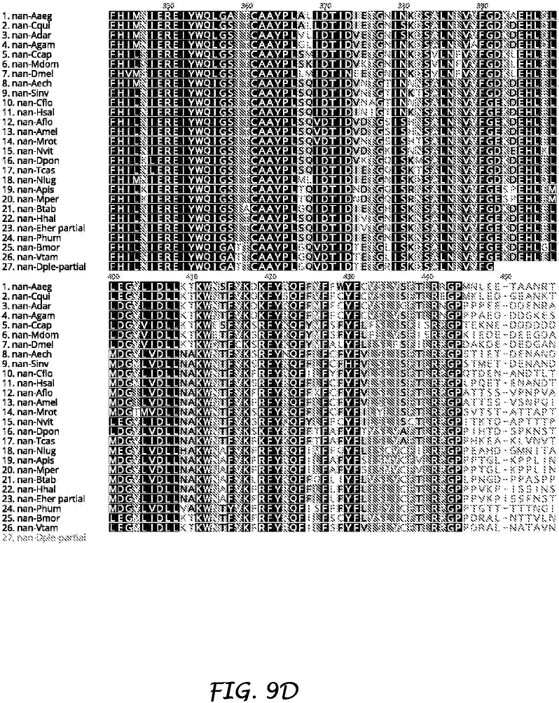

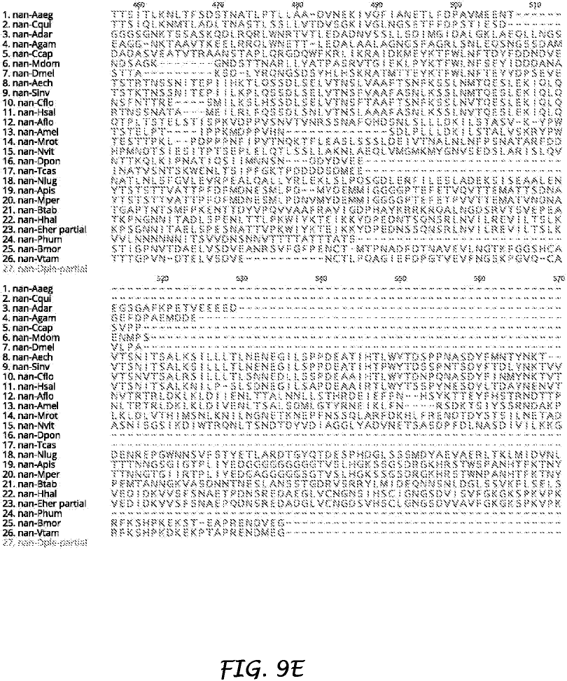

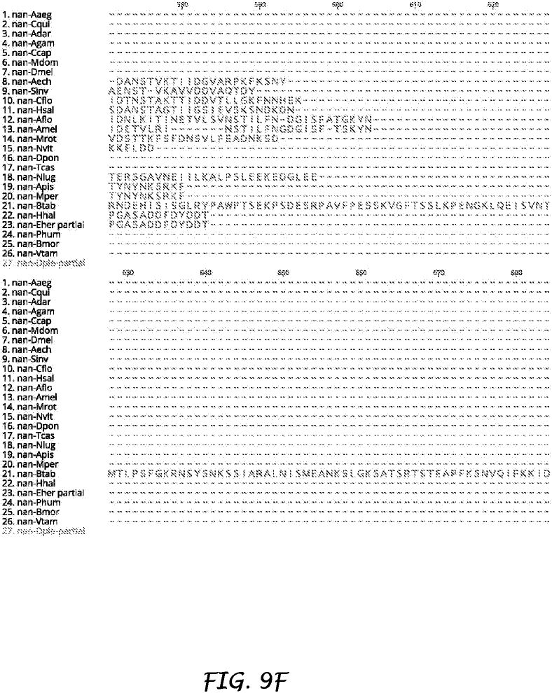

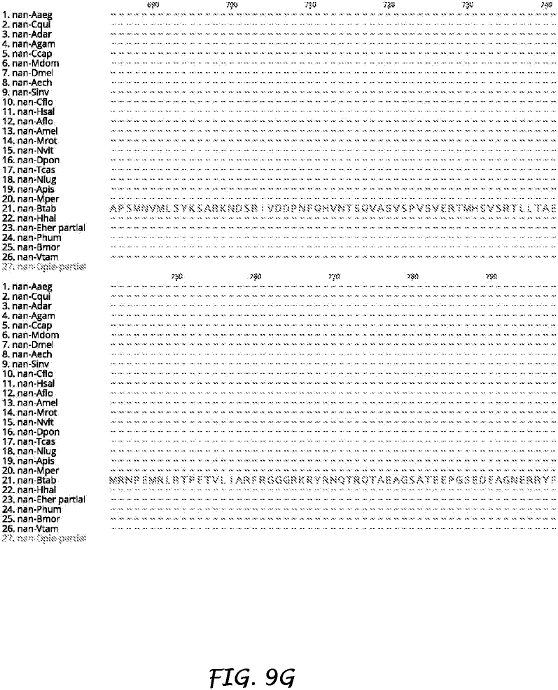

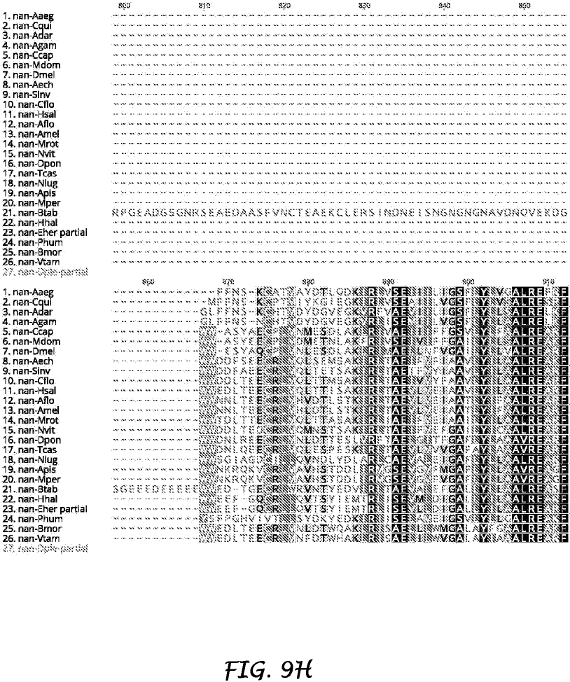

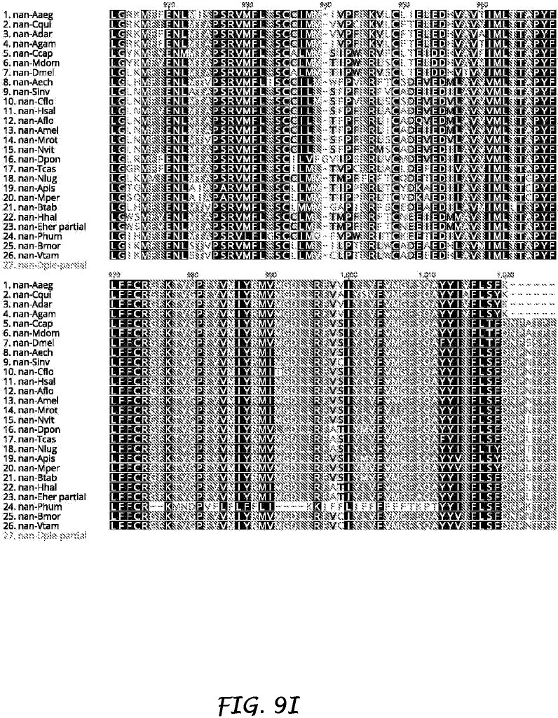

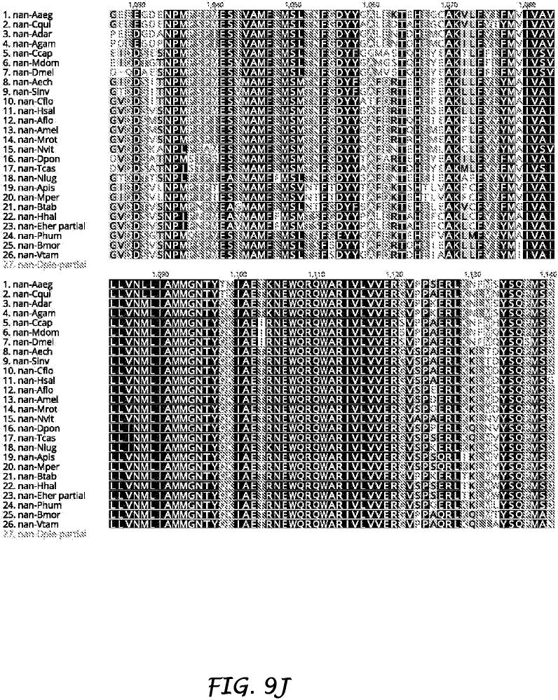

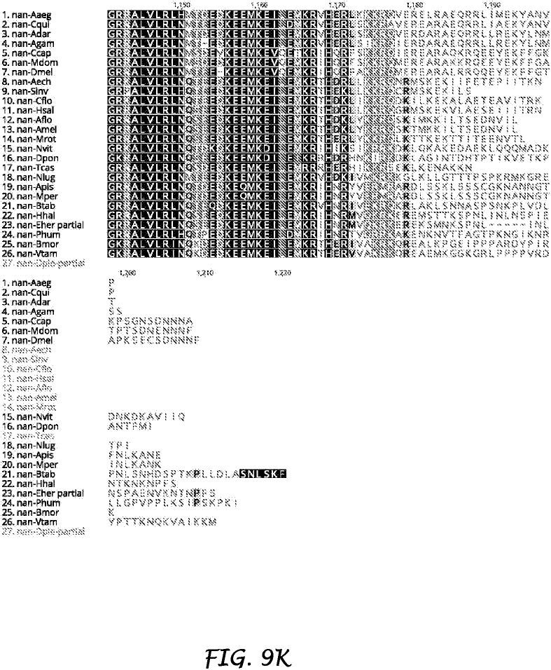

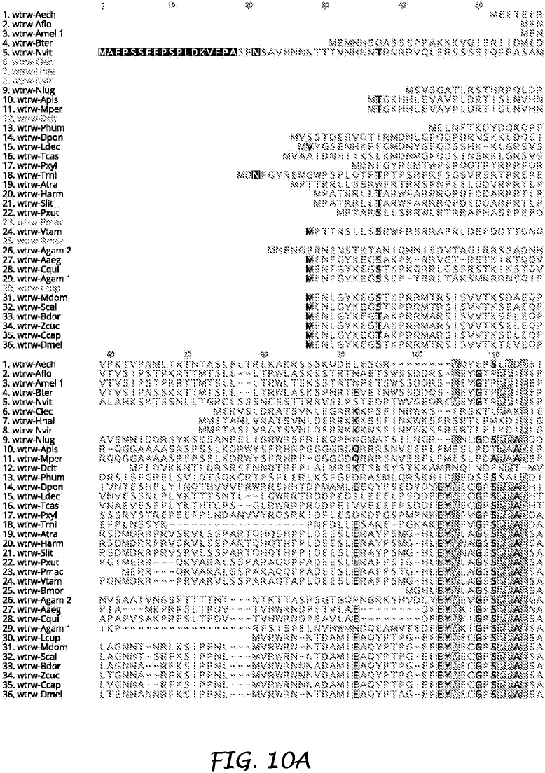

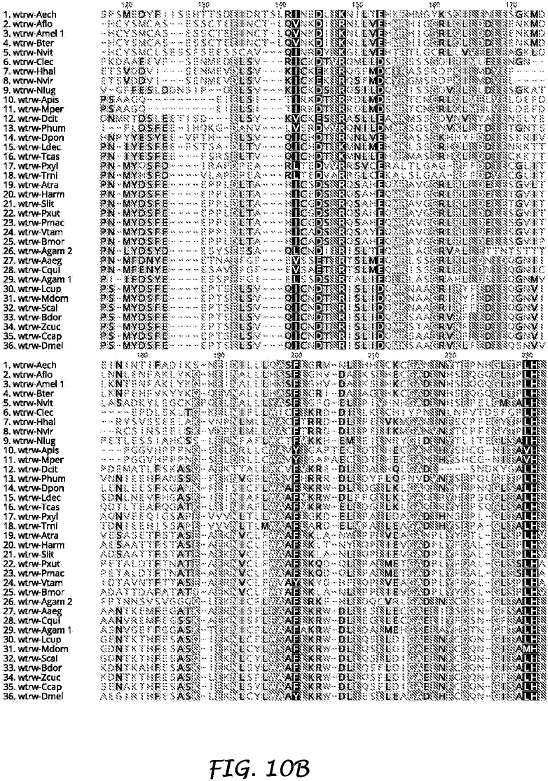

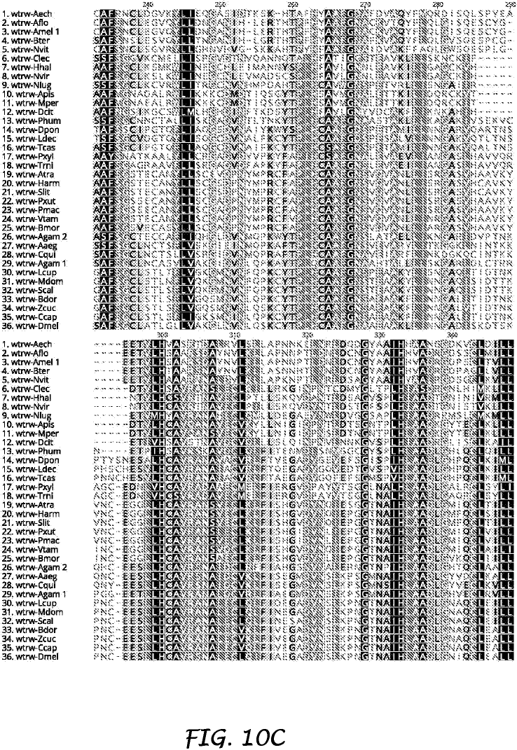

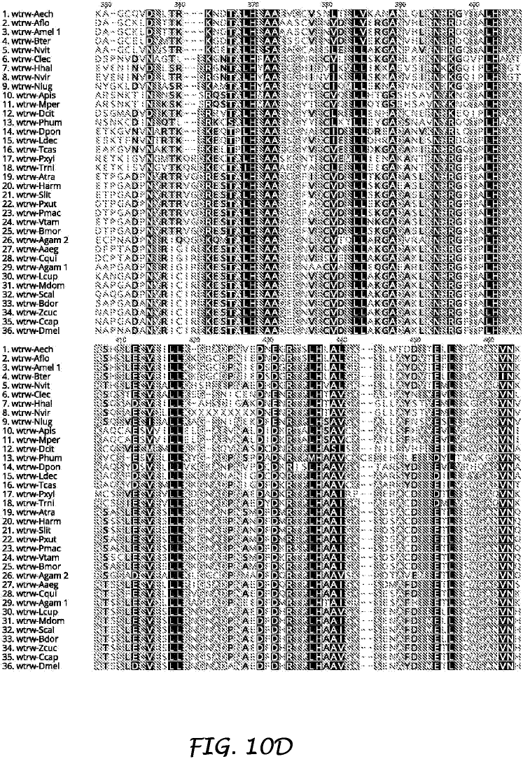

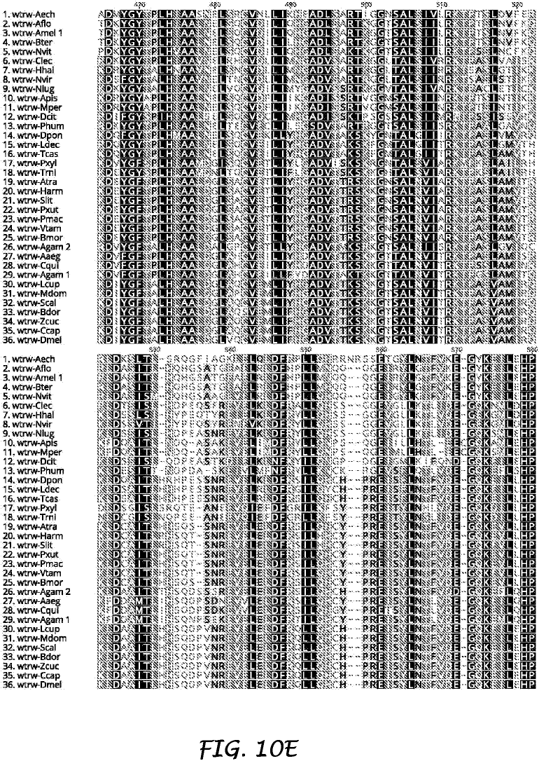

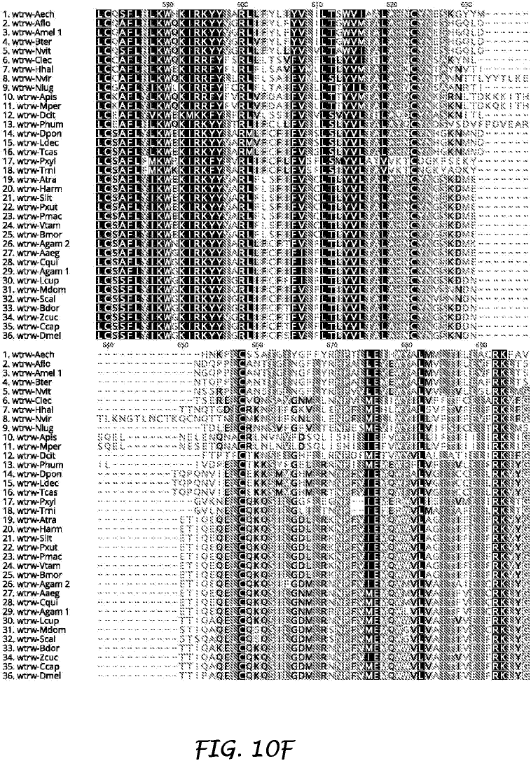

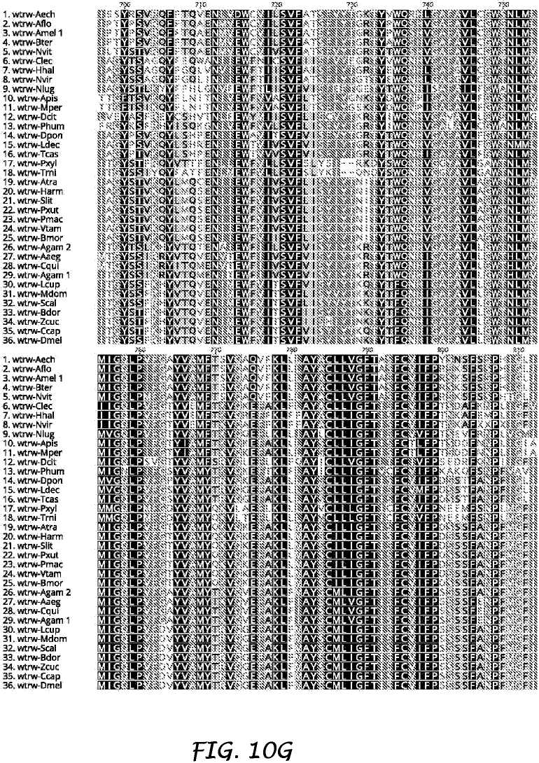

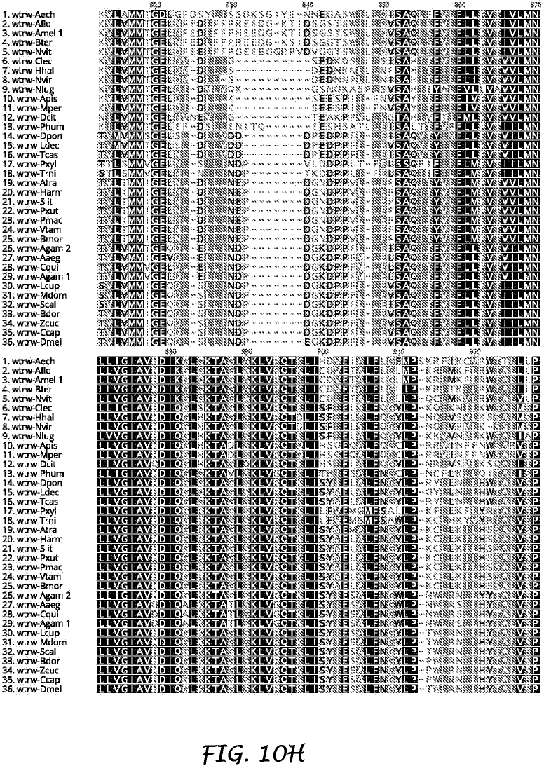

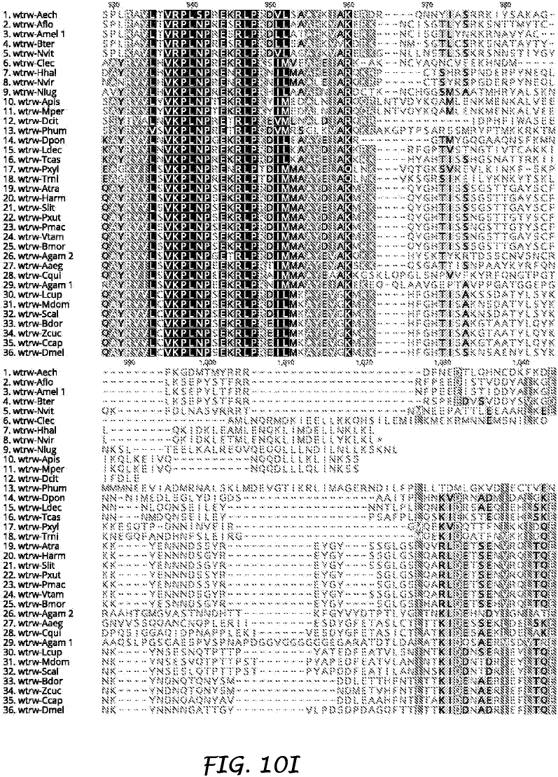

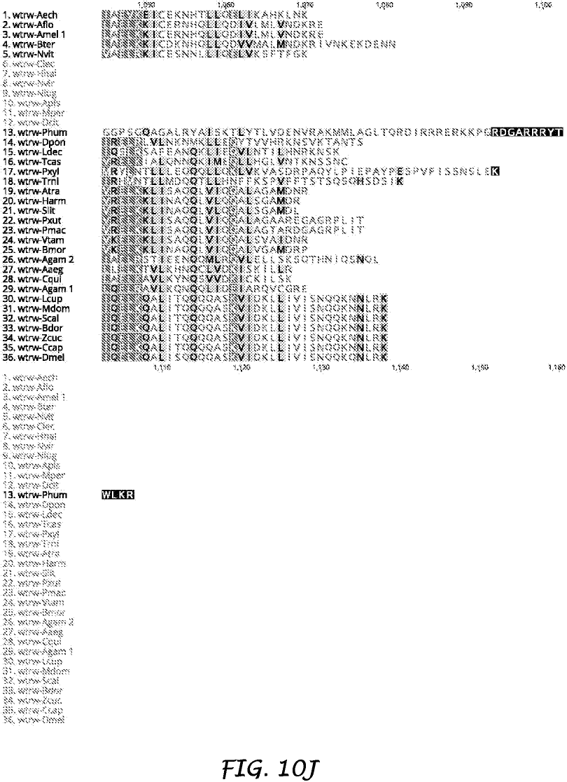

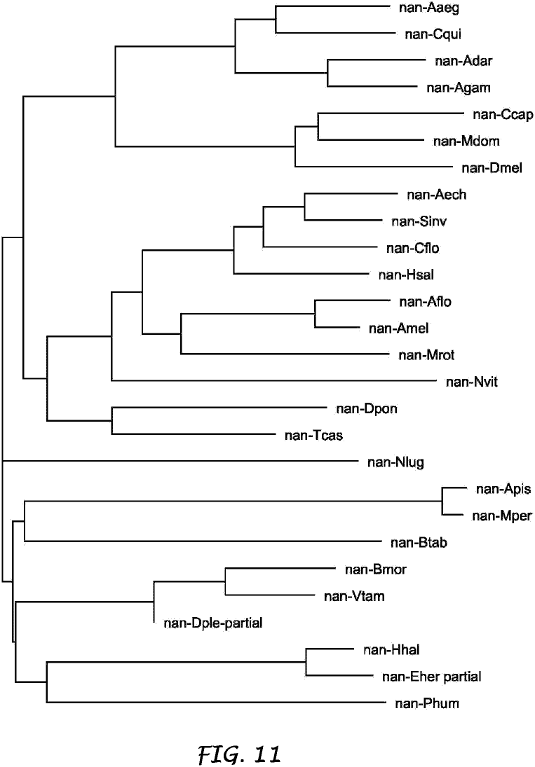

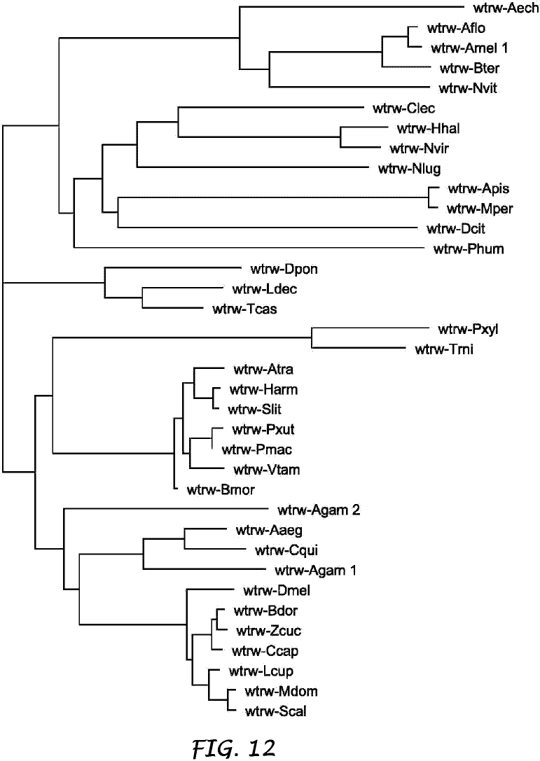

TRPA channel Water with and TRPV cahnnel Inactive belong to different subfamilies and that Water witch has only 22% identity to Inactive (FIG. 8). Yet, the inventors have discovered that TRPV protein Nanchung forms a complex with TRPA protein Water witch, analogous to previously described complex between TRPV protein Nanchung and TRPV protein Inactive.

Furthermore, the inventors have discovered that protein complex formed by TRPV protein Nanchung and TRPA protein Water witch can be modulated by commercial insecticides afidopyropen and pymetrozine analogous to the complex formed by TRPV proteins Nanchung and Inactive.

Even further, the inventors have discovered that protein complex formed by TRPV protein Nanchung and TRPA protein Water witch can be modulated by endogenous agonist nicotinamide analogous to the complex formed by TRPV proteins Nanchung and Inactive.

Water witch protein has been predicted not to interact with electrophilic plant-derived insecticides and/or repellents cinnamaldehyde and allyl isothiocyanate (Kang et al., 2010). Yet the inventors have discovered that Water witch can be modulated cinnamaldehyde and allyl isothiocyanate.

Even further the inventors have discovered that formation of complex between TRPV protein Nanchung and TRPA protein Water witch greatly increases binding affinity of the commercial insecticide afidopyropen.

Even further the inventors have discovered that commercial insecticides afidopyropen and pymetrozine and endogenous agonist nicotinamide bind to the same site in the complex comprising TRPV protein Nanchung and TRPV protein Inactive.

Even further the inventors have discovered that commercial insecticides afidopyropen and pymetrozine and endogenous agonist nicotinamide bind to the same site in the complex comprising TRPV protein Nanchung and TRPA protein Water witch.

Furthermore, the inventors have developed methods of identifying additional compounds that bind directly and modulate the complex comprising TRPV protein Nanchung and TRPA protein Water witch.

Even further, the inventors have developed methods of identifying additional compounds that directly modulate Water witch protein or complexes formed by Nanchung protein and Water witch protein.

Identification of such compounds may lead to the identification of suitable chemical compounds that can function either as insecticides or insect repellents and be useful in both pest managemen programs, plant protection, crop protection, and public health.

### II. Definitions

Unless defined otherwise, all technical and scientific terms used herein have the same meaning as commonly understood to one of ordinary skill in the art. Although any methods, devices, and materials similar or equivalent to those described herein can be used in the practice or testing of the presently disclosed subject matter, representative methods, devices, and materials are now described.

The terms “a,” “an,” and “the” refer to “one or more” when used in this application, including the claims. Thus, for example, reference to “a cell” (e.g., “a mammalian cell”) includes a plurality of such cells (e.g., a plurality of mammalian cells in culture).

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The terms “insect,” “insect pest,” or “plant insect pest” refer to any one of the numerous usually small arthropod animals of the class Insecta that are known to associate with plants and which, as a result of that association, causes a detrimental effect on the plant’s health and vigor. The term plant as used herein encompasses whole plants and parts of plants such as roots, stems, leaves and seed, as well as cells and tissues within the plants or plant parts. The terms “insect,” “insect pest,” or “plant insect pest” are used interchangeably throughout the instant application.

The terms “pests” and/or “pest species” refer to any one of numerous usually small arthropod animals of the class Insecta that are known to associate with plants and which, as a result of that association, causes a detrimental effect on the plant’s health and vigor. Additionally, the terms refer to include mollusk, nematodes, other arthropod classes that are known to associate with plants and which, as a result of that association, causes a detrimental effect on the plant’s health and vigor. The terms further includes arthropods, gastropods and nematodes including but not limited to: insects from the order of Lepidoptera, for example Achroia grisella, Acleris spp. such as A. fimbriana, A. gloverana, A. variana; Acrolepiopsis assectella, Acronicta major, Adoxophyes spp. such as A. cyrtosema, A. orana; Aedia leucomelas, Agrotis spp. such as A. exclamationis, A. fucosa, A. ipsilon, A. orthogoma, A. segetum, A. subterranea; Alabama argillacea, Aleurodicus dispersus, Alsophila pometaria, Ampelophaga rubiginosa, Amyelois transitella, Anacampsis sarcitella, Anagasta kuehniella, Anarsia lineatella, Anisota senatoria, Antheraea pernyi, Anticarsia (=Thermesia) spp. such as A. gemmatalis; Apamea spp, Aproaerema modicella, Archips spp. such as A. argyrospila, A. fuscocupreanus, A. rosana, A. xyloseanus; Argyresthia conjugella, Argyroploce spp., Argyrotaenia spp. such as A. velutiNanchunga; Athetis mindara, Austroasca viridigrisea, Autographa gamma, Autographa nigrisigna, Barathra brassicae, Bedellia spp., Bonagota salubricola, Borbo cinnara, Bucculatrix thurberiella, Bupalus piniarius, Busseola spp., Cacoecia spp. such as C. muriNanchunga, C. podana; Cactoblastis cactorum, Cadra cautella, Calingo braziliensis, Caloptilis theivora, Capua reticulana, Carposina spp. such as C. niponensis, C. sasakii; Cephus spp, Chaetocnema aridula, Cheimatobia brumata, Chilo spp. such as C. Indicus, C. suppressalis, C. partellus; Choreutis pariana, Choristoneura spp. such as C. conflictana, C. fumiferana, C. longicellana, C. muriNanchunga, C. occidentalis, C. rosaceana; Chrysodeixis (=Pseudoplusia) spp. such as C. eriosoma, C. includens; Cirphis unipuncta, Clysia ambiguella, Cnaphalocerus spp., Cnaphalocrocis medinalis, Cnephasia spp., Cochylis hospes, Coleophora spp., Colias eurytheme, Conopomorpha spp., Conotrachelus spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinicola, Dendrolimus spp. such as D. pini, D. spectabilis, D. sibiricus; Desmia funeralis, Diaphania spp. such as D. nitidalis, D. hyalinata; Diatraea grandiosella, Diatraea saccharalis, Diphthera festiva, Earias spp. such as E. insulana, E. vittella; Ecdytolopha aurantianu, Egira (=Xylomyges) curialis, Elasmopalpus lignosellus, Eldana saccharina, Endopiza viteana, Ennomos subsignaria, Eoreuma loftini, Ephestia spp. such as E. cautella, E. elutella, E. kuehniella; Epinotia aporema, Epiphyas postvittana, Erannis tiliaria, Erionota thrax, Etiella spp., Eulia spp., Eupoecilia ambiguella, Euproctis chrysorrhoea, Euxoa spp., Evetria bouliana, Faronta albilinea, Feltia spp. such as F. subterranean; Galleria mellonella, Gracillaria spp., Grapholita spp. such as G. funebrana, G. molesta, G. inopinata; Halysidota spp., Harrisina americana, Hedylepta spp., Helicoverpa spp. such as H. armigera (=Heliothis armigera), H. zea (=Heliothis zea); Heliothis spp. such as H. assulta, H. subflexa, H. virescens; Hellula spp. such as H. undalis, H. rogatalis; Helocoverpa gelotopoeon, Hemileuca oliviae, Herpetogramma licarsisalis, Hibernia defoliaria, Hofmannophila pseudospretella, Homoeosoma electellum, Homona magNanchungima, Hypena scabra, Hyphantria cunea, Hyponomeuta padella, Hyponomeuta malinellus, Kakivoriaflavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma salicis, Leucoptera spp. such as L. coffeella, L. scitella; Leuminivora lycinivorella, Lithocolletis blancardella, Lithophane antennata, Llattia octo (=Amyna axis), Lobesia botrana, Lophocampa spp., Loxagrotis albicosta, Loxostege spp. such as L. sticticalis, L. cereralis; Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanitis leda, Mocis spp. such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosalis, Operophtera brumata, Orgyia pseudotsugata, Oria spp., Orthaga thyrisalis, Ostrinia spp. such as O. nubilalis; Oulema oryzae, Paleacrita vernata, Panolis flammea, Parnara spp., Papaipema nebris, Papilio cresphontes, Paramyelois transitella, Paranthrene regalis, Paysandisia archon, Pectinophora spp. such as P. gossypiella; Peridroma saucia, Perileucoptera spp., such as P. coffeella; Phalera bucephala, Phryganidia californica, Phthorimaea spp. such as P. operculella; Phyllocnistis citrella, Phyllonorycter spp. such as P. blancardella, P. crataegella, P. issikii, P. ringoniella; Pieris spp. such as P. brassicae, P. rapae, P. napi; Pilocrocis tripunctata, Plathypena scabra, Platynota spp. such as P. flavedana, P. idaeusalis, P. stultana; Platyptilia carduidactyla, Plebejus argus, Plodia interpunctella, Plusia spp, Plutella maculipennis, Plutella xylostella, Pontia protodica, Prays spp., Prodenia spp., Proxenus lepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festaliella, Scirpophaga spp. such as S. incertulas, S. innotata; Scotia segetum, Sesamia spp. such as S. inferens, Seudyra subflava, Sitotroga cerealella, Sparganothis pilleriana, Spilonota lechriaspis, S. ocellana, Spodoptera (=Lamphygma) spp. such as S. eridania, S. exigua, S. frugiperda, S. latisfascia, S. littoralis, S. litura, S. omithogalli; Stigmella spp., Stomopteryx subsecivella, Strymon bazochii, Sylepta derogata, SyNanchungthedon spp. such as S. exitiosa, Tecia solanivora, Telehin licus. Thaumatopoea pityocampa, Thaumatotibia (=Cryptophlebia) leucotreta, Thaumetopoea pityocampa, Thecla spp., Theresimima ampelophaga, Thyrinteina spp, Tildenia inconspicuella, Tinea spp. such as T. cloacella, T. pellionella; Tineola bisselliella, Tortrix spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp, Yponomeuta padella, and Zeiraphera canadensis;insects from the order of Coleoptera, for example Acalymma vittatum, Acanthoscehdes obtectus, Adoretus spp., Agelastica alni, Agrilus spp. such as A. anxius, A. planipennis, A. sinuatus; Agriotes spp. such as A. fuscicollis, A. lineatus, A. obscurus; Alphitobius diaperinus, Amphimallus solstitialis, Anisandrus dispar, Anisoplia austriaca, Anobium punctatum, Anomala corpulenta, Anomala rufocuprea, Anoplophora spp. such as A. glabripennis; Anthonomus spp. such as A. eugenii, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophorafemoralis, Blastophagus piniperda, Blitophaga undata, Bruchidius obtectus, Bruchus spp. such as B. lentis, B. pisorum, B. rufimanus; Byctiscus betulae, Callidiellum rufipenne, Callopistria floridensis, Callosobruchus chinensis, Cameraria ohridella, Cassida nebulosa, Cerotoma trifurcata, Cetonia aurata, Ceuthorhynchus spp. such as C. assimilis, C. napi; Chaetocnema tibialis, Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp, Dactylispa balyi, Dectes texanus, Dermestes spp., Diabrotica spp. such as D. undecimpunctata, D. speciosa, D. longicornis, D. semipunctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. similaris; Eutheola humilis, Eutinobothrus brasiliensis, Faustinus cubae, Gibbium psylloides, Gnathocerus cornutus, Hellula undalis, Heteronychus arator, Hylamorpha elegans, Hylobius abietis, Hylotrupes bajulus, Hypera spp. such as H. brunneipennis, H. postica; Hypomeces squamosus, Hypothenemus spp., Ips typographus, Lachnosterna consanguinea, Lasioderma serricorne, Latheticus oryzae, Lathridius spp., Lema spp. such as L. bilineata, L. melanopus; Leptinotarsa spp. such as L. decemlineata; Leptispa pygmaea, Limonius californicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp, Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. helleri; Phyllotreta spp. such as P. chrysocephala, P. nemorum, P. striolata, P. vittula; Phyllopertha horticola, Popillia japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. billineatus, R. ferrugineus, R. palmarum, R. phoenicis, R. vulneratus; Saperda candida, Scolytus schevyrewi, Scyphophorus acupunctatus, Sitona lineatus, Sitophilus spp. such as S. granaria, S. oryzae, S. zeamais; Sphenophorus spp. such as S. levis; Stegobium paniceum, Sternechus spp. such as S. subsignatus; Strophomorphus ctenotus, Symphyletes spp., Tanymecus spp., Tenebrio molitor, Tenebrioides mauretanicus, Tribolium spp. such as T. castaneum; Trogoderma spp., Tychius spp., Xylotrechus spp. such as X. pyrrhoderus; and, Zabrus spp. such as Z. tenebrioides; insects from the order of Diptera for example Aedes spp. such as A. aegypti, A. albopictus, A. vexans; Anastrepha ludens, Anopheles spp. such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria; Chrysops atlanticus, Chrysops discalis, Chrysops silacea, Cochliomyia spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus; Culicoides furens, Culiseta inornata, Culiseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzukii, Fannia spp. such as F. canicularis; Gastraphilus spp. such as G. intestinalis; Geomyza tipunctata, Glossina spp. such as G. fuscipes, G. morsitans, G. palpalis, G. tachinoides; Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprina, L. cuprina, L. sericata; Lycoria pectoralis, Mansonia titillanus, Mayetiola spp. such as M. destructor; Musca spp. such as M. autumnalis, M. domestica; Muscina stabulans, Oestrus spp. such as O. ovis; Opomyza florum, Oscinella spp. such as O. frit; Orseolia oryzae, Pegomya hysocyami, Phlebotomus argentipes, Phorbia spp. such as P. antiqua, P. brassicae, P. coarctata; Phytomyza gymnostoma, Prosimulium mixtum, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalis; Simulium vittatum, Sitodiplosis mosellana, Stomoxys spp. such as S. calcitrans; Tabanus spp. such as T. atratus, T. bovinus, T. lineola, T. similis; Tannia spp., Thecodiplosis japonensis, Tipula oleracea, Tipula paludosa, and Wohlfahrtia spp.; insects from the order of Thysanoptera for example, Baliothrips biformis, Dichromothrips corbetti, Dichromothrips ssp., Echinothrips americanus, Enneothrips flavens, Frankliniella spp. such as F. fusca, F. occidentalis, F. tritici; Heliothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp. such as S. citri, S. dorsalis, S. perseae; Stenchaetothrips spp, Taeniothrips cardamoni, Taeniothrips inconsequens, Thrips spp. such as T. imagines, T. hawaiiensis, T. oryzae, T. palmi, T. parvispinus, T. tabaci; insects from the order of Hemiptera for example, Acizzia jamatonica, Acrosternum spp. such as A. hilare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani, Aleurocanthus woglumi, Aleurodes spp., Aleurodicus disperses, Aleurolobus barodensis, Aleurothrixus spp., Amrasca spp., Anasa tristis, Antestiopsis spp., Anuraphis cardui, Aonidiella spp., Aphanostigma piri, Aphidula nasturtii, Aphis spp. such as A. craccivora, A. fabae, A. forbesi, A. gossypii, A. grossulariae, A. maidiradicis, A. pomi, A. sambuci, A. schneideri, A. spiraecola; Arboridia apicalis, Arilus critatus, Aspidiella spp., Aspidiotus spp., Atanus spp., Aulacaspis yasumatsui, Aulacorthum solani, Bactericera cockerelli (Paratrioza cockerelli), Bemisia spp. such as B. argentifolii, B. tabaci (Aleurodes tabaci); Blissus spp. such as B. leucopterus; Brachycaudus spp. such as B. cardui, B. helichrysi, B. persicae, B. prunicola; Brachycolus spp., Brachycorynella asparagi, Brevicoryne brassicae, Cacopsylla spp. such as C. fulguralis, C. pyricola (Psylla piri); Calligypona marginata, Calocoris spp., Campylomma livida, Capitophorus horni, Carneocephala fulgida, Cavelerius spp., Ceraplastes spp., Ceratovacuna lanigera, Ceroplastes ceriferus, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as C. hemipterus, C. lectularius; Coccomytilus halli, Coccus spp. such as C. hesperidum, C. pseudomagnoliarum; Corythucha arcuata, Creontiades dilutus, Cryptomyzus ribis, Chrysomphalus aonidum, Cryptomyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citri; Diaspis spp. such as D. bromeliae; Dichelops furcatus, Diconocoris hewetti, Doralis spp., Dreyfusia nordmannianae, Dreyfusia piceae, Drosicha spp., Dysaphis spp. such as D. plantaginea, D. pyri, D. radicola; Dysaulacorthum pseudosolani, Dysdercus spp. such as D. cingulatus, D. intermedius; Dysmicoccus spp., Edessa spp, Geocoris spp, Empoasca spp. such as E. fabae, E. solana; Epidiaspis leperii, Eriosoma spp. such as E. lanigerum, E. pyricola; Erythroneura spp., Eurygaster spp. such as E. integriceps; Euscelis bilobatus, Euschistus spp. such as E. heros, E. impictiventris, E. servus; Fiorinia theae, Geococcus coffeae, Glycaspis brimblecombei, Halyomorpha spp. such as H. halys; Heliopeltis spp., Homalodisca vitripennis (=H. coagulata), Horcias nobilellus, Hyalopterus pruni, Hyperomyzus lactucae, Icerya spp. such as I. purchase; Idiocerus spp., Idioscopus spp., Laodelphax striatellus, Lecanium spp., Lecanoideus floccissimus, Lepidosaphes spp. such as L. ulmi; Leptocorisa spp., Leptoglossus phyllopus, Lipaphis erysimi, Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadrilineatus, Mahanarva fimbriolata, Megacopta cribraria, Megoura viciae, Melanaphis pyrarius, Melanaphis sacchari, Melanocallis (=Tinocallis) caryaefoliae, Metcafiella spp., Metopolophium dirhodum, Monellia costalis, Monelliopsis pecanis, Myzocallis coryli, Murgantia spp, Myzus spp. such as M. ascalonicus, M. cerasi, M. nicotianae, M. persicae, M. varians; Nasonovia ribis-nigri, Neotoxoptera formosana, Neomegalotomus spp, Nephotettix spp. such as N. malayanus, N. nigropictus, N. parvus, N. virescens; Nezara spp. such as N. viridula; Nilaparvata lugens, Nysius huttoni, Oebalus spp. such as O. pugnax; Oncometopia spp., Orthezia praelonga, Oxycaraenus hyalinipennis, Parabemisia myricae, Parlatoria spp., Parthenolecanium spp. such as P. corni, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinii, Phorodon humuli, Phylloxera spp. such as P. devastatrix, Piesma quadrata, Piezodorus spp. such as P. guildinii; Pinnaspis aspidistrae, Planococcus spp. such as P. citri, P. ficus; Prosapia bicincta, Protopulvinaria pyriformis, Psallus seriatus, Pseudacysta persea, Pseudaulacaspis pentagona, Pseudococcus spp. such as P. comstocki; Psylla spp. such as P. mali; Pteromalus spp., Pulvinaria amygdali, Pyrilla spp., Quadraspidiotus spp., such as Q. perniciosus; Quesada gigas, Rastrococcus spp., Reduvius senilis, Rhizoecus americanus, Rhodnius spp., Rhopalomyzus ascalonicus, Rhopalosiphum spp. such as R. pseudobrassicas, R. insertum, R. maidis, R. padi; Sagatodes spp., Sahlbergella singularis, Saissetia spp., Sappaphis mala, Sappaphis mali, Scaptocoris spp, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Spissistilus festinus (=Stictocephala festina); Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp. such as T. aurantii; Trialeurodes spp. such as T. abutilonea, T. ricini, T. vaporariorum; Triatoma spp., Trioza spp., Typhlocyba spp., Unaspis spp. such as U. citri, U. yanonensis; and Viteus vitifolii; insects from the order Hymenoptera for example Acanthomyops interjectus, Athalia rosae, Atta spp. such as A. capiguara, A. cephalotes, A. cephalotes, A. laevigata, A. robusta, A. sexdens, A. texana, Bombus spp., Brachymyrmex spp., Camponotus spp such as C. floridanus, C. pennsylvanicus, C. modoc; Cardiocondyla nuda, Chalibion sp, Crematogaster spp., Dasymutilla occidentalis, Diprion spp., Dolichovespula maculata, Dorymyrmex spp, Dryocosmus kuriphilus, Formica spp, Hoplocampa spp. such as H. minuta, H. testudinea; Iridomyrmex humilis, Lasius spp. such as L. niger, Linepithema humile, Liometopum spp, Leptocybe invasa, Monomorium spp such as M. pharaonis, Monomorium, Nylandria fulva, Pachycondyla chinensis, Paratrechina longicornis, Paravespula spp such as P. germanica, P. pennsylvanica, P. vulgaris; Pheidole spp such as P. megacephala; Pogonomyrmex spp such as P. barbatus, P. californicus, Polistes rubiginosa, Prenolepis impairs, Pseudomyrmex gracilis, Schelipron spp, Sirex cyaneus, Solenopsis spp such as S. geminata, S.invicta, S. molesta, S. richteri, S. xyloni, Sphecius speciosus, Sphex spp, Tapinoma spp such as T. melanocephalum, T. sessile; Tetramorium spp such as T. caespitum, T. bicarinatum, Vespa spp. such as V. crabro; Vespula spp such as V. squamosal; Wasmannia auropunctata, Xylocopa sp.; insects from the order Orthoptera for example Acheta domesticus, Calliptamus italicus, Chortoicetes terminifera, Ceuthophilus spp, Diastrammena asynamora, Dociostaurus maroccanus, Gryllotalpa spp such as G. africana, G. gryllotalpa; Gryllus spp, Hieroglyphus daganensis, Kraussaria angulifera, Locusta spp. such as L. migratoria, L. pardalina; Melanoplus spp such as M. bivittatus, M. femurrubrum, M. mexicanus, M. sanguinipes, M. spretus; Nomadacris septemfasciata, Oedaleus senegalensis, Scapteriscus spp, Schistocerca spp such as S. americana, S. gregaria, Stemopelmatus spp, Tachycines asynamorus, and Zonozerus variegatus; pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentor spp such as D.silvarum, D. andersoni, D. variabilis, Hyalomma spp. such as H. truncatum, Ixodes spp. such as I. ricinus, I. rubicundus, I. scapularis, I. holocyclus, I. pacificus, Rhipicephalus sanguineus, Ornithodorus spp. such as O. moubata, O. hermsi, O. turicata), Ornithonyssus bacoti, Otobius megnini, Dermanyssus gallinae, Psoroptes spp such as P. ovis, Rhipicephalus spp such as R. sanguineus, R. appendiculatus, Rhipicephalus evertsi), Rhizoglyphus spp; Sarcoptes spp. such asS. Scabiei; and Family Eriophyidae including Aceria spp such as A. sheldoni, A. anthocoptes, Acallitus spp; Aculops spp. such as A. lycopersici, A. pelekassi; Aculus spp such as A. schlechtendali; Colomerus vitis, Epitrimerus pyri, Phyllocoptruta oleivora; Eriophytes ribis and Eriophyes spp such as Eriophyes sheldoni; Family Tarsonemidae including Hemitarsonemus spp., Phytonemus pallidus and Polyphagotarsonemus latus, Stenotarsonemus spp. Steneotarsonemus spinki; Family Tenuipalpidae including Brevipalpus spp. such as B. phoenicis; Family Tetranychidae including Eotetranychus spp., Eutetranychus spp., Oligonychus spp., Petrobia latens, Tetranychus spp such as T. cinnabarinus, T. evansi, T. kanzawai, T, pacificus, T. phaseulus, T. telarius and T. urticae; Bryobia praetiosa; Panonychus spp. such as P. ulmi, P. citri;, Metatetranychus spp. and Oligonychus spp. such as O. pratensis, O. perseae), Vasates lycopersici; Raoiella indica, Family Carpoglyphidae including Carpoglyphus spp; Penthaleidae spp such as Halotydeus destructor; Family Demodicidae with species such a Demodex spp; Family Trombicidea including Trombicula spp:; Family Macronyssidae including Ornothonyssus spp; Family Pyemotidae including Pyemotes tritici; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa; pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifolii; Seed gall nematodes, Anguina spp.; Stem and foliar nematodes, Aphelenchoides spp. such as A. besseyi; Sting nematodes, Belonolaimus spp. such as B. longicaudatus; Pine nematodes, Bursaphelenchus spp. such as B. lignicolus, B. xylophilus; Ring nematodes, Criconema spp.; Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Heliocotylenchus multicinctus; Sheath and sheathoid nematodes, Hemicycliophora spp. and Hemicriconemoides spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. claytoni, T. dubius; Citrus nematodes, Tylenchulus spp. such as T. semipenetrans; Dagger nematodes, Xiphinema spp.; and other plant parasitic nematode species; insects from the order Isoptera for example Calotermesflavicollis, Coptotermes spp such as C. formosanus, C. gestroi, C. acinaciformis; Cornitermes cumulans, Cryptotermes spp such as C. brevis, C. cavifrons; Globitermes sulfureus, Heterotermes spp such as H. aureus, H. longiceps, H. tenuis; Leucotermes flavipes, Odontotermes spp., Incisitermes spp such as I. minor, I. Snyder; Marginitermes hubbardi, Mastotermes spp such as M. darwiniensis Neocapritermes spp such as N. opacus, N. parvus; Neotermes spp, Procornitermes spp, Zootermopsis spp such as Z. angusticollis, Z. nevadensis, Reticulitermes spp. such as R. hesperus, R. tibialis, R. speratus, R. flavipes, R. grassei, R. lucifugus, R. santonensis, R. virginicus; Termes natalensis; insects from the order Blattaria for example Blatta spp such as B. orientalis, B. lateralis; Blattella spp such as B. asahinae, B. germanica; Leucophaea maderae, Panchlora nivea, Periplaneta spp such as P. americana, P. australasiae, P. brunnea, P. fuligginosa, P. japonica; Supella longipalpa, Parcoblatta pennsylvanica, Eurycotis floridana, Pycnoscelus surinamensis; insects from the order Siphonoptera for example Cediopsylla simples, Ceratophyllus spp., Ctenocephalides spp such as C. felis, C. canis, Xenopsylla cheopis, Pulex irritans, Trichodectes canis, Tunga penetrans, and Nosopsyllus fasciatus; insects from the order Thysanura for example Lepisma saccharina, Ctenolepisma urbana, and Thermobia domestica; pests from the class Chilopoda for example Geophilus spp., Scutigera spp. such as Scutigera coleoptrata; pests from the class Diplopoda for example Blaniulus guttulatus, Julus spp, Narceus spp.; pests from the class Symphyla for example Scutigerella immaculata; insects from the order Dermaptera, for example Forficula auricularia; insects from the order Collembola, for example Onychiurus spp. such as Onychiurus armatus; pests from the order Isopoda for example, Armadillidium vulgare, Oniscus asellus, Porcellio scaber; insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.. Examples of further pest species which may be controlled by the compounds and/or compositions identified herein include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulusfilaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis Nanchunga, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

The terms “non-pest” and/or “non-target” species refers to all other animals that would not be classified as “pests” and/or “pest species” as defined above.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides, conservatively modified variants thereof, complementary sequences, and degenerate codon substitutions that have similar binding properties as the reference nucleic acid and are metabolized in a manner similar to naturally occurring nucleotides.

The terms “nucleic acid” or “nucleic acid molecule” can also be used interchangeably with gene, open reading frame (ORF), cDNA, and mRNA encoded by a gene, nucleic acid molecule, nucleic acid fragment, nucleic acid segment, or polynucleotide.

The term “gene” is used broadly to refer to any segment of nucleic acid associated with a biological function. Thus, genes include coding sequences, and/or the regulatory sequences required for their expression. For example, “gene” refers to a nucleic acid fragment that expresses mRNA, functional RNA, or specific protein, including regulatory sequences. “Genes” also include non-expressed DNA segments that, for example, form recognition sequences for other proteins. “Genes” can be obtained from a variety of sources, including cloning from a source of interest or synthesizing from known or predicted sequence information, and may include sequences designed to have desired parameters.

The term “gene delivery” or “gene transfer” refers to methods or systems for reliably introducing foreign DNA into target cells and include injection, transduction, transfection, transformation and electroporation. Such methods can result in transient or long-term expression of genes.

The term “transduction” refers to the delivery of a DNA molecule to a recipient cell either in vivo or in vitro, via a replication-defective viral vector, such as, e.g., adenoviral vector.

The term “transfection” is used to refer to the uptake of foreign DNA by a mammalian cell. A cell has been “transfected” when exogenous DNA has been introduced across the cell plasma membrane. Transfection can be used to introduce one or more exogenous DNA moieties, such as a plasmid vector and other nucleic acid molecules, into suitable cells. The term refers to both stable and transient uptake of the genetic material.

The term “transformation” and/or “transfection” refers to a process for introducing heterologous DNA into a cell. Transformed cells are understood to encompass not only the end product of a transformation/transfection process, but also transgenic progeny thereof.

The term “injection” refers to a process for introducing heterologous DNA or RNA into a cell using a syringe and a needle.

The term “vector” refers to any genetic element, such as a plasmid, phage, transposon, cosmid, chromosome, virus, virion, etc., which is capable of replication when associated with the proper control elements, such as a helper virus, and which can transfer gene sequences between cells. Thus, the term includes cloning and expression vehicles, as well as replication-defective viral vectors.

The terms “expression vector” and “expression cassette” refer to a nucleic acid molecule capable of directing expression of a particular nucleotide sequence in an appropriate host cell, typically comprising a promoter operatively linked to the nucleotide sequence of interest which is operatively linked to termination signals. It also typically comprises sequences required for proper translation of the nucleotide sequence. The coding region usually encodes a polypeptide(s) of interest but can also encode a functional RNA of interest, for example antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombiNanchungt form useful for heterologous expression. Typically, however, the expression cassette is heterologous with respect to the host; i.e., the particular DNA sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant, the promoter can also be specific to a particular tissue, organ, or stage of development.

The term “adenovirus” refers to a vector derived from an adenovirus serotype. Adenoviruses are double-stranded DNA viruses capable of infecting broad range of mammalian cells, both dividing and non-dividing. The adenovirus DNA is linear of approximately 36,000 bp. The most widely used adenoviral vectors for gene delivery are the replication-deficient vectors, in which the E1 and E3 regions of the adenoviral genome are deleted. E1-deleted viruses are propagated in complementing cells, such as HEK-293, which provide E1-encoded proteins in trans. E1/E3-deleted adenoviruses can accommodate up to 7.5 kb of foreign DNA. The classical method of incorporating foreign DNA into adenovirus genome involves homologous recombination in mammalian cells. More efficient method, as described in the present examples involves homologous recombination in Escherichia coli between a large plasmid containing most of the adenovirus genome and a small shuttle plasmid. The shuttle plasmid contains the expression cassette flanked by sequences homologous to the region to be targeted in the viral genome. The recombiNanchungt Ad genome is then linearized by restriction digestion and used to transfect E1-complementing mammalian cells to produce viral particles (Douglas JT., Methods Mol Biol. 2004; V246:3-14) To produce adenoviruses containing toxic gene, special adenoviral vectors have been developed where expression of foreign gene is suppressed during virus production. One of these systems, named, SpeAd Ad-teto system is offered by, for example, Welgen, Inc.

The term “tetracycline-controlled transactivator” (tTA) refers to a fusion protein used to control nucleic acid expression in the presence or absence of doxyclycline, tetracycline and related compounds. The tTA includes a Tet repressor (TetR) fused to any domain capable of activating transcription. The tTA may include a TetR fused to a C-terminal portion of adenovirus.

The term “operably linked” refers to two nucleic acid sequences that are related physically or functionally. For example, a promoter or regulatory DNA sequence is said to be “operably linked to” a DNA sequence that encodes an RNA or a polypeptide if the two sequences are situated such that the regulatory DNA sequence will affect the expression level of the coding or structural DNA sequence. A promoter is also said to be operably linked to a nucleotide sequence if when an RNA polymerase binds to the promoter under conditions sufficient for transcription, the nucleotide sequence is transcribed.

The term “isolated,” when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high performance liquid chromatography. A polypeptide that is the predominantly Nanchung species present in a preparation is substantially isolated. The term “isolated” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 99% pure.

The terms “label” and “labeled” refer to the attachment of a moiety, capable of detection by spectroscopic, radiologic, or other methods, to a molecule. Thus, the terms “label” or “labeled” refer to incorporation or attachment, optionally covalently or non-covalently, of a detectable marker into a molecule, such as a biomolecule. Various methods of labeling biomolecules are known in the art and can be used. Examples of labels for biomolecules include, but are not limited to, the following: radioisotopes, fluorescent labels, heavy atoms, enzymatic labels or reporter genes, chemiluminescent groups, and biotinyl groups. In some embodiments, labels are attached by spacer arms of various lengths to reduce potential steric hindrance. Fluorescent probe that can be utilized include, but are not limited to fluorescein isothiocyanate; fluorescein dichlorotriazine and fluorinated analogs of fluorescein; naphthofluorescein carboxylic acid and its succinimidyl ester; carboxyrhodamine 6G; pyridyloxazole derivatives; Cy2, 3, 3.5, 5, 5.5, and 7; phycoerythrin; phycoerythrin-Cy conjugates; fluorescent species of succinimidyl esters, carboxylic acids, isothiocyanates, sulfonyl chlorides, and dansyl chlorides, including propionic acid succinimidyl esters, and pentanoic acid succinimidyl esters; succinimidyl esters of carboxytetramethylrhodamine; rhodamine Red-X succinimidyl ester; Texas Red sulfonyl chloride; Texas Red-X succinimidyl ester; Texas Red-X sodium tetrafluorophenol ester; Red-X; Texas Red dyes; tetramethylrhodamine; lissamine rhodamine B; tetramethylrhodamine; tetramethylrhodamine isothiocyanate; naphthofluoresceins; coumarin derivatives (e.g., hydroxycoumarin, aminocoumarin, and methoxycoumarin); pyrenes; pyridyloxazole derivatives; dapoxyl dyes; Cascade Blue and Yellow dyes; benzofuran isothiocyanates; sodium tetrafluorophenols; 4,4-difluoro-4-bora-3a,4a-diaza-s-indacene; Alexa fluors (e.g., 350, 430, 488, 532, 546, 555, 568, 594, 633, 647, 660, 680, 700, and 750); isotope of hydrogen (e.g., tritium isotope, 3H), isotope of carbon (e.g., 14C) green fluorescent protein; yellow fluorescent protein or red fluorescent protein. The peak excitation and emission wavelengths will vary for these compounds and selection of a particular fluorescent probe for a particular application can be made in part based on excitation and/or emission wavelengths.

As used herein, the terms “candidate compound” or “test compound” refer to the collection of compounds that are to be screened for their ability to bind to Nanchung proteins, Water witch protein, Nanchung and Inactive protein complexes, Nanchung and Water witch protein complexes and/or modulate insect TRPV channels and/or insect TRPA channels. The candidate compounds may encompass numerous classes of chemical molecules, e.g., small organic or inorganic molecules, polysaccharides, biological macromolecules, e.g., peptides, proteins, peptide analogs and derivatives, peptidomimetics, nucleic acids, nucleic acid analogs and derivatives, an extract made from biological materials such as bacteria, plants, fungi, or animal cells or tissues, naturally occurring or synthetic compositions, or combinations thereof. Generally, the candidate compounds can have a molecular weight of about 50 to 500,000, but is not limited thereto.

As used herein, the term “small molecule” refers to a compound that is “natural product-like,” however, the term “small molecule” is not limited to a “natural product-like” compound. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

In some embodiments, the candidate compound may be a synthetic molecule. The term “synthetic molecule” refers to a molecule that does not occur in nature.

In certain embodiments, the candidate compound may be a naturally-occurring molecule. Such a naturally-occurring molecule may be used in a purified or unpurified form, i.e., as obtained from the biological source. The term “naturally occurring” refers to an entity (e.g., a cell, biomolecule, etc.) that is found in nature as distinct from being artificially produced by man. For example, a polypeptide or nucleotide sequence that is present in an organism in its natural state, which has not been intentionally modified or isolated by man in the laboratory, is naturally occurring. As such, a polypeptide or nucleotide sequence is considered “non-naturally occurring” if it is encoded by or present within a recombiNanchungt molecule, even if the amino acid or nucleic acid sequence is identical to an amino acid or nucleic acid sequence found in nature.

Depending upon the particular embodiment being practiced, the candidate compounds may be provided free in solution, or may be attached to a carrier, or a solid support, e.g., beads. A number of suitable solid supports may be employed for immobilization of the candidate compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, the candidate compounds may be screened individually, or in groups. Group screening is particularly useful where hit rates for effective candidate compounds are expected to be low such that one would not expect more than one positive result for a given group.

There are millions of possible candidate compounds. Methods for developing small molecule, polymeric and genome-based libraries are known and described, for example, in Ding, et al. J. Am. Chem. Soc. 124: 1594-1596 (2002) and Lynn, et al., J. Am. Chem. Soc. 123: 8155-8156 (2001). A number of small molecule libraries are known in the art and commercially available. Commercially available compound libraries can be obtained from, e.g., ArQule, Pharmacopia, graffinity, Panvera, Vitas-M Lab, Biomol International and Oxford. These libraries can be screened using the screening methods described herein. Chemical compound libraries such as those from of 10,000 compounds and 86,000 compounds from NIH Roadmap, Molecular Libraries Screening Centers Network (MLSCN) can also be used. A comprehensive list of compound libraries can be found at www.broad.harvard.edu/chembio/platform/screening/compound_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

The term “modulate” refers to an increase, decrease, or other alteration of any, or all, chemical and/or biological activities and/or properties of a biomolecule, such as the TRPV or TRPA (e.g., insect TRPV or TRPA) channel of the present invention. The term “modulation” as used herein thus refers to both upregulation (i.e., activation or stimulation) and downregulation (i.e., inhibition or suppression) of such an activity or property. As would be understood by one of ordinary skill in the art, a modulation of a chemical and/or biological activity and/or property of a biomolecule, such as TRPV or TRPA channel, can result from an increase or decrease in the expression of the biomolecule in a cell. Accordingly, the terms “modulate” and grammatical variants thereof are intended to encompass both direct modulation (e.g., inhibition of a chemical and/or biological activity and/or property of a polypeptide via binding of an inhibitor to the polypeptide) as well as indirect modulation (e.g., upregulation or downregulation of expression of a protein, such as a TRPV or TRPA channel or inhibition or stimulation of a biomolecule that acts together with a biomolecule of the presently disclosed subject matter to produce a biological effect).

The term “endogenous modulator” refers to the modulator originating or produced within an organism, tissue, or cell.

The term “exogenous modulator” refers to the modulator originating or produced from outside organism, tissue, or cell.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “TRP channel” refers to a channel and/or a protein complex formed from one or more of the following: Nanchung protein and/or Water witch protein and/or Inactive protein. It is to be understood that the TRPV channel and/or TRPV protein complex is a type of TRP channel and/or TRP protein complex. Nanchung proteins and Inactive proteins are a type of TRPV proteins which are a type of TRP proteins. Water witch protein is a type of TRPA proteins which are a type of TRP proteins. TRP proteins may be found in insect cellular membranes; mammalian cellular membranes; derived from a free cell system; produced by a cell culture; and/or produced by cells, including but not limited to a frog egg, insect cell, and/or mammalian cell, that have been genetically modified (including either or both DNA and/or RNA) to include the genes coding for one or more of the Nanchung proteins, Water witch proteins and/or the Inactive proteins. It is to be understood that the TRP proteins disclosed herein may be mammalian TRP proteins and/or arthropod TRP proteins and/or Nematode TRP proteins and/or Mollusk TRP proteins. It is to be further understood that Nanchung, Inactive and Water witch proteins may be found in arthropods, including, but not limited to insects, as well as Mollusks and/or Nematodes. It is to be understood that TRP and/or TRPV and/or TRPA channels may be formed from one or more TRPA and/or TRPV protein complexes; from two or more TRPA and/or TRPV protein complexes; from three or more TRPA and/or TRPV protein complexes, and/or from four or more TRPA and/or TRPV protein complexes.

The term “binding affinity” refers to the ability of compounds to form a bond with a receptor. For purposes of this invention, an equilibrium constant is used to determine the binding affinity of one or more compounds with the one or more Nanchung proteins, Inactive proteins, Water witch proteins, TRP channels and/or TRPV channels and/or TRPA channels. A dissociation constant (Kd) is a specific type of equilibrium constant measured in a labeled compound saturation binding assay. It is the molar concentration of labeled compound that binds to half the receptor sites at equilibrium. The lower the dissociation constant the higher the binding affinity of the candidate compound for the Nanchung protein, Inactive protein, Water witch protein and/or complexes formed by Nanchung and Inactive proteins and/or complexes formed by Nancung and Water witch proteins. An inhibition constant (Ki) is a specific type of equilibrium constant that is measured using competition binding of an unlabeled compound with a labeled compound. It is the molar concentration of the competing unlabeled compound that would occupy 50% of the receptors at equilibrium, if no labeled compound was present. It can be calculated using the Cheng and Prusoff equation: Kᵢ=IC₅₀/(1+[L]/K_{d}) where IC₅₀ is the molar concentration of competing unlabeled compound which reduces the specific binding of a labeled compound by 50%. In this equation [L] is the molar concentration of a labeled compound in the assay; Kd is equilibrium dissociation constant for a labeled compound. In addition to saturation binding assay, equilibrium dissociation constant, Kd can be measured in multiple ways, including but not limited to:
1. Using the association and dissociation rates constants where Kd=Koff/Kon
2. Using a fluorescence polarization assay
3. Using surface plasmon resonance
4. Using thermophoresis
5. Using isothermal calorimetry
6. Using Schild analysis
Surface plasmon resonance and isothermal calorimetry do not require labeled compound to measure K_{d}. In addition, for the competition assay, K_{d} and/or Ki (for competition assay) may be calculated using one or more of the following: Cheng and Prusoff equation, fluorescence polarization, and/or Schild analysis. It is to be understood that the K_{d} and Ki values calculated for a compound should be similar, but due to experimental error the values may have less than a two-fold difference between the two values or less than a three-fold difference between the two-values depending upon the extent of the experimental error.

The term “high affinity” and/or “high binding affinity” refers to a compound whose equilibrium dissociation constant (K_{d}) and/or equilibrium inhibition constant (Ki) is calculated as being less than or equal to 3x10⁻⁷ M, less than or equal to 3x10⁻⁸ M, and/or less than or equal to 30x10⁻¹² M. The lower the K_{d} or Kᵢ the higher the binding affinity for the compound.

The term “mode of action” refers to the classification of a compound and/or a composition according to the Insecticide Resistance Action Committee (IRAC) into the appropriate group based on the compound and/or composition (a) exhibiting the same function as other insecticides in that group and (b) binding directly to the same site on particular proteins, protein complexes and/or protein channels as other compounds and/or compositions in that group.

The term “insecticide” refers to a substance or agent used to kill insects and other arthropods.

The term “repellent” refers to a substance or agent used to repel insects and other arthropods.

### III. Screening Methods

The inventors have discovered that insect TRPA Water witch proteins and insect TRPV Nanchung proteins physically associate with each other and form complexes consisting of one or more TRPV Nanchung proteins and one or more TRPA Water witch proteins.

Furthermore, the inventors have discovered that complexes consisting of one or more TRPA Water witch proteins and one or more TRPV Nanchung proteins can form ion channels. As such, in one embodiment, the present invention relates to a method for determining whether or not a candidate compound is a modulator of an insect TRPA Water witch channels and/or complexes consisting of TRPA Water witch channels and TRPV Nanchung channels.

Furthermore, the inventors have discovered that ion channels formed by TRPA Water witch proteins and/or ion channels formed by one or more TRPA Water witch proteins and one or more TRPV Nanchung proteins may be used to identify compounds that kill insects and/or disrupt insect behavior and/or repel insects and thus be useful as insecticides and/or repellents.

Furthermore, the inventors have discovered that determining whether or not a candidate compound is a modulator of an insect TRPA Water witch channels and/or complexes consisting of TRPA Water witch channels and TRPV Nanchung channels may assist with the classification of a compound’s mode of action.

Furthermore, the inventors have determined that a compound, afidopyropen, binds with high affinity to protein complexes consisting of one or more TRPV Nanchung proteins and one or more TRPA Water witch proteins.

The inventors have further discovered methods of identifying compounds that bind directly to the complexes formed by one or more TRPV Nanchung proteins and one or more TRPA Water witch proteins.

Furthermore, the inventors have found that methods of identifying compounds that bind directly to the complexes formed by one or more TRPV Nanchung proteins and one or more TRPA Water witch proteins may be used to identify compounds that kill insects and/or disrupt insect behavior and/or repel insects and thus be useful as insecticides and/or repellents.

Furthermore, the inventors have discovered that determining whether or not different compounds bind to the same or different sites of the complexes consisting of TRPA Water witch protein and TRPV Nanchung protein may assist with the classification of a compound’s mode of action.

The methods identified herein includes providing one or more Nanchung proteins, and/or one or more Water witch proteins contacting the first cell with a candidate compound; and assaying for modulation of the ion channel formed by TRPA Water witch protein and/or ion channels comprising one or more TRPV Nanchung proteins and one or more TRPA Water witch protein. Modulation of the ion channel formed by TRPA Water witch protein and/or ion channels comprising one or more TRPV Nanchung proteins and one or more TRPA Water witch protein may be assayed using conventional in vitro and in vivo methods well known to the skilled artisan. For example, in certain embodiments, the assaying step may include (1) detecting calcium ion mobilization in the first cell in response to the candidate compound, (2) detecting a membrane potential in the first cell in response to the candidate compound, (3) comparing calcium ion mobilization in the first cell in the absence of the candidate compound with calcium ion mobilization in the first cell in the presence of the candidate compound. For example, calcium response may be measured by assessment of the uptake of Ca²⁺ or by using fluorescent dyes, such as Fluo-4 or Fura-2 or fluorescent proteins, such as Cameleon or GCaMP6m, (4) comparing a membrane potential in the first cell in the absence of the candidate compound with a membrane potential in the first cell in the presence of the candidate compound, (5) comparing calcium ion mobilization in the first cell in the presence of the candidate compound with calcium ion mobilization reference level indicative of no modulation of the of the ion channel formed by TRPA Water witch protein and/or ion channels comprizing one or more TRPV Nanchung proteins and one or more TRPA Water witch protein, and/or (6) comparing a membrane potential in the first cell in the presence of the candidate compound with a membrane potential reference level indicative of no modulation of the ion channel formed by TRPA Water witch protein and/or ion channels comprising one or more TRPV Nanchung proteins and one or more TRPA Water witch protein (7) comparing ion currents in the first cell in the absence of the candidate compound with a ion currents in the first cell in the presence of the candidate compound using standard electrophysiology methods, such as voltage clamp or patch clamp. It is to be understood that this same assay could be run on insect TRPV and/or TRPA channels from vertebrate, arthropod, mollusks, nematodes or any other organisms. Such a test would allow one to determine whether or not the candidate compound worked as a modulator is those systems and thus, help one determine the potential environmental impact of using the candidate compound as an insecticide.

Preferably, the candidate compound may modulate the calcium ion mobilization or the membrane potential, or ion currents in the first cell by at least 10% relative to the reference level; more preferably at least 20% relative to the reference level; more preferably at least 30% relative to the reference level; more preferably at least 40% relative to the reference level; more preferably at least 50% relative to the reference level; more preferably at least 60% relative to the reference level; more preferably at least 70% relative to the reference level; more preferably at least 75% relative to the reference level; more preferably at least 80% relative to the reference level; more preferably at least 85% relative to the reference level; more preferably at least 90% relative to the reference level; and more preferably at least 95% relative to the reference level.

In addition, modulation of ion channels formed by TRPA Water witch protein and/or ion channels comprising one or more TRPV Nanchung proteins and one or more TRPA Water witch protein may be assayed using a variety of other conventional assays that measure changes in ion fluxes including, but not limited to, patch clamp techniques, measurement of whole cell currents, radiolabeled ion flux assays, and fluorescence assays using voltage sensitive dyes (Zheng et al., 2004).

Modulation of ion channels formed by TRPA Water witch protein and/or ion channels comprising one or more TRPV Nanchung proteins and one or more TRPA Water witch protein may also be assessed using a variety of other in vitro and in vivo assays to determine functional, chemical, and physical effects, e.g., measuring the binding of the insect insect TRPV and/or TRPA channels to other molecules, including peptides, small organic molecules, and lipids; measuring insect TRPV and/or TRPA protein and/or mRNA levels, or measuring other aspects of insect TRPV and/or TRPA polypeptides, e.g., transcription levels, or physiological changes.

The inventors have further determined that it is beneficial to identify compounds that bind directly to complexes formed by one or more TRPV Nanchung proteins and one or more TRPA Water witch protein and thus be useful as insecticides or repellents.

There are two variations of binding assays, a saturation assay and a competition assay. The saturation assay may include at least one of the following steps: (1) contacting the one or morecomplexes formed by Nanchung and Water witch proteins with a labeled compound; (2) measuring theamount of the labeled compound which is bound to the one or more complexes formed by Nanchung and Water witch proteins; (3) calculating the equilibrium inhibitor constant dissociation constant (K_{d}) for the labeled compound (4) wherein the K_{d} may be determined using a (a) maximum binding (Bₘₐₓ) and (b) the concentration of the compound needed to reach 50% of maximum binding.

The competition assay may include one or more of the following steps: (1) contacting the one or more complexes formed by Nanchung and Water witch proteins with two compounds, wherein one compound is labeled and another compound is unlabeled; (2) .measuring the amount of the labeled compound which is bound to the one or more complexes formed by Nanchung and Water witch proteins at single concentration of labeled compound and various concentrations of unlabeled compound; (3) determining the concentration of the unlabeled compound which produces 50% maximal binding of labeled compound (IC₅₀); (4) calculating the equilibrium inhibitory constant (Ki) for the unlabeled compound; (4) wherein Ki can be determined from (a) IC50 of unlabeled compound, (b) Kd of labeled compound and (c) concentration of labeled compound.

The competition binding assay also determines whether labeled and unlabeled compounds bind to the same or different parts of the complexes formed by Nanchung and Water witch proteins.

Binding to the complexes formed by Nanchung and Water witch proteins may also be determined using one or more of the following methods: (I) thermoforesis (II) fluorescence polarization assay, (III) surface plasmon resonance, and (IV) isothermal calorimetry.

Identification of molecular targets of insecticides is essential for assessing their spectra, health and environmental impacts, as well as for insect resistance management. Pymetrozine (PM) and afidopyropen (AP) ) are two commercial insecticides which act by disrupting coordination and feeding of plant-sucking insects such as aphids and whiteflies leading to death caused by starvation and dessication (Casida and Durkin, 2013; Maienfisch, 2012).

The inventors have previously shown that PM and AP act by directly binding protein Nanchung, a member of vanniloid-type (TRPV) ion channels (Kandasamy et al., 2017) Binding to Nanchung protein alone, however, was not sufficient to modulate the ion channels. Biological activity of PM and AP required simultaneous presence of another member of TRPV proteins, Inactive (Kandasamy et al., 2017; Nesterov et al., 2015). Together Nanchung and Inactive formed a functional complex, which responded to PM and AP by calcium mobilization and depolarization of plasma membrane. Moreover, whrereas PM and AP could bind Nanchung in the absence of Inactive, the presence of Inactive greatly increased the binding affinity (Kandasamy et al., 2017).

The current invention shows that analogous to TRPV ion channel Inactive, ankyrin-type (TRPA) channel, Water witch can form a functional complex with TRPV channel Nanchung. The current invention also found that complexes formed by Nanchung and Water witch proteins can be activated by PM and AP similarly to complexes formed by Nanchung and Inactive proteins. The inventors also found that complexes formed by both Nanchung and Inactive proteins and Nanchung and Water witch proteins can be activated by the endogenous modulator, nicotinamide. The inventors also found that analogous to co-expression of Inactive together with Nanchung, co-expression of Water witch together with Nanchung greatly potentiates binding of afidopyropen to Nanchung. As such, the inventors found that with regard to both function and binding complexes formed by Nanchung and Water witch proteins are functionally similar to complexes formed by Nanchung and Inactive proteins. This finding was unexpected because Inactive and Water witch belong to different types of TRP channels, vanilloid-type and ankyrin-type, respectively. Furthemore, based on amino acid sequence alignment Water witch shares only 22% sequence identity with Inactive (FIG. 8). The inventors also found that endogenous modulator nicotinamide and exogenous modulators AP and PM have overlapping binding sites. The inventors also found that channels formed by Whater witch protein and channels formed by Nanchung and Water witch proteins can be activated by plant-derived compounds cinnamaldehyde and allyl isothiocyanate which can act as insecticides and/or insect repellents. This finding was unexpected because the main target of these compounds is thought to be insect TRPA1 protein. Furhemore, based on amino acid sequence, Water witch protein has been predicted not to be a target of cinnamaldehyde and allyl isothiocyanate (Kang et al., 2010) Thus, the current invention identifies methods of determining whether or not a candidate compound modulates activity of Water witch protein and/or complexes formed by Nanchung protein and Water witch protein. Identifying such modulators allows for identification of new insecticides and/or insect repellents. Furthermore, the current invention identifies methods of detecting binding, calculating binding affinity and determining whether or not a candidate compounds binds directly to the same or different sites on complexes formed by Nanchung and Water witch proteins. Determining the binding location and affinities for candidate compounds allows for identification of new insecticides and/or insect repellents, assists with the classification of a compounds mode of action and allows for better insect management and control that may result in decreased risk of developing resistant insect populations.

As noted previously, the candidate compound may be a small organic molecule, small inorganic molecule, polysaccharides, peptides, proteins, nucleic acids, an extract made from biological materials such as bacteria, plants, fungi, animal cells, animal tissues, and any combination thereof.

Generally, a candidate compound can be tested at any concentration that can modulate the activity of a ion channel formed by Water witch protein, and/or activity of ion channel formed by complexes comprising one or more Nanchung protein and one or more Water witch protein, and/or exhibit binding to complexes comprising one or more Nanchung protein and one or more Water witch protein. In some embodiments, the candidate compound may be tested at a concentration in the range of about 0.01 nM to about 1000 mM. In certain other embodiments, the compound may be tested in the range of about 100 µM to about 1000 µM. In certain further embodiments, the candidate compound may be tested at greater than 0.05 mM, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.0 mM, or 2 mM. Other ranges and concentrations of the candidate compound may also be suitable.

In some embodiments, the candidate compound may be tested at two or more different concentrations. Preferably the highest concentration tested is at least 2×, at least 3×, at least 4×, at least 5×, at least 6×, at least 7×, at least 8×, at least 9×, at least 10×, at least 15×, at least 20×, at least 25×, at least 50×, at least 75×, at least 100×, at least 200×, at least 250× higher than the lowest concentration employed. For example, the candidate compound may be tested at 0.1 mM, 0.5 mM, and 1 mM, where the highest concentration is at least 10X the concentration of the lowest concentration employed.

Generally, the one or more Water witch protein, and/or complexes formed by Nanchung protein and Water witch protein may be contacted with a candidate compound for any suitable length of time before measuring the binding affinity and/or activity of the Water witch protein, and/or complexes comprising one or more Nanchung protein and one or more Water witch protein. For example, Water witch protein, and/or complexes formed by Nanchung protein and Water witch protein may be contacted with a candidate compound for at least 5 seconds, at least 10 seconds, at least 15 seconds, at least 30 seconds, at least 45 seconds, at least 1 minute, at least 2 minutes, at least 3 minutes, at least 4 minutes, at least 5 minutes, at least 10 minutes, at least 15 minutes, at least 30 minutes, at least 60 minutes, at least 2 hours, at least 3 hours or more before activity of Water witch protein, and/or complexes formed by Nanchung protein and Water witch protein is measured. In some embodiments, activity may be measured at the instant when Water witch protein, and/or complexes formed by Nanchung protein and Water witch protein are contacted with a candidate compound.

In some embodiments, the binding affinity and/or activity of Water witch protein, and/or complexes formed by Nanchung protein and Water witch protein may be measured over a period of time. For example, activity may be measured for a period of at least 5 seconds, at least 10 seconds, at least 15 seconds, at least 30 seconds, at least 45 seconds, at least 1 minute, at least 2 minutes, at least 3 minutes, at least 4 minutes, at least 5 minutes, at least 10 minutes, at least 15 minutes, at least 30 minutes, at least 60 minutes, at least 2 hours, at least 3 hours, at least 4 hours or more. The measurement period can start before the Water witch protein, and/or complexes formed by Nanchung protein and Water witch protein are contacted with a candidate compound, at the instant when Water witch protein, and/or complexes formed by Nanchung protein and Water witch protein are first contacted with a candidate compound or start after a period of time after the one or more Water witch protein, and/or complexes formed by Nanchung protein and Water witch protein are first contacted with a candidate compound. The Water witch protein, and/or complexes formed by Nanchung protein and Water witch protein may be continuously contacted with the candidate compound while activity and/or binding is measured.

In some embodiments, the candidate compound has an effective concentration EC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM for activating and/or binding Water witch protein, and/or complexes formed by Nanchung protein and Water witch protein.

In some embodiments, the candidate compound has an IC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM for inhibiting and/or binding Water witch protein, and/or complexes formed by Nanchung protein and Water witch protein.

In certain embodiments, the candidate compound is a modulator that inhibits the activity of Water witch protein, and/or complexes formed by Nanchung protein and Water witch protein.

In certain other embodiments, the candidate compound is a modulator that activates the Water witch protein, and/or complexes formed by Nanchung protein and Water witch protein.

Preferably, the candidate compound is a modulator that perturbs an insect behavior and/or kills insects and/or repels insects.

In certain embodiments the candidate compound binds directly to the complexes formed by Nanchung protein and Water witch protein

Preferably the candidate compound modulates ion channels formed by Water witch protein or ion channels formed by Nanchung protein and Water witch protein.

In certain embodiments, Water witch protein and/or complexes formed by Nanchung protein and Water witch protein is in a biological cell. The term “biological cell” or “cell” as used herein has its commonly understood meaning. Inside a cell, Water witch protein, and/or complexes formed by Nanchung protein and Water witch protein may be expressed from an endogenous gene in the cell or from at least one vector that is transfected into the cell. It is to be understood that either the cultured cell, or a cell obtained directly from animal, such as frog oocyte, or whole tissue may be used as a source of Water witch protein and/or complexes formed by Nanchung protein and Water witch protein. Preferably, Nanchung and Water witch proteins are co-expressed in the cell. The Nanchung and Water witch proteins may be co-expressed at varying rations.

In certain embodiments, Water witch protein and/or complexes formed by Nanchung protein and Water witch protein are formed in a cell-free system. The term “cell-free system” as used herein means an in vitro system used to study biological reactions that happen within cells while reducing the complex interactions found in a whole cell. Subcellular fractions can be isolated by ultracentrifugation to provide molecular machinery that can be used in reactions in the absence of many of the other cellular components. Cell-free biosystems can be prepared by mixing one or more of the following: ribosomes; and/or DNA and/or RNA encoding for one or more proteins of interest; membranes; as well as a one or more enzymes and/or coenzymes. Cell-free biosystems have several advantages suitable in industrial applications. In vitro biosystems can be easily controlled. Cell-free systems provide an alternative choice for fast protein synthesis and very high product yields are usually accomplished without the formation of by-products or synthesis of cell mass.

In certain embodiments a cell genetically modified to contain the genes coding for one or more of the Nanchung protein, and/or Water witch protein.

TRPV protein Nanchung and TRPA protein Water witch may be used to produce ion channels for purposes of the methods described herein.

In certain embodiments a nucleic acid coding for one or more TRPV protein Nanchung and/or TRPA protein Water witch be introduced into frog oocytes or other cells (parental cells) to produce the TRPA and/or TRPV channel complex for purposes of the methods described herein.

The insect TRPV and/or TRPA protein complex and/or channel used in the screening assay of the present invention may be any insect TRPV and/or TRPA protein complex and/or channel or homolog or a conservative variant thereof. For example, the TRPV and/or TRPA protein complex and/or channel may be a Drosophila TRPV and/or other member of TRPA channel superfamily. Other sources of TRPV and/or TRPA protein complex and/or channel may also be suitable for use according to the methods of the present invention.

In some embodiments, the Nanchung nucleic acid sequences may be selected from the group consisting of a) a polynucleotide molecule comprising a nucleic acid molecule having a sequence selected from the group consisting of SEQ ID NOS: 1-27; b) a polynucleotide molecule having at least about 70% sequence identity to the sequence of SEQ ID NOS: 1-27; and c) a fragment of the polynucleotide molecule of a) or b). Such fragments can be a UTR, a core promoter, an intron, an enhancer, a cis-element, or any other regulatory element.

In some embodiments, the Water witch nucleic acid sequences may be selected from the group consisting of a) a polynucleotide molecule comprising a nucleic acid molecule having a sequence selected from the group consisting of SEQ ID NOS: 28-63; b) a polynucleotide molecule having at least about 70% sequence identity to the sequence of SEQ ID NOS: 28-63; and c) a fragment of the polynucleotide molecule of a) or b). Such fragments can be a UTR, a core promoter, an intron, an enhancer, a cis-element, or any other regulatory element.

The disclosed polynucleotides are capable of providing for expression of Nanchung and Water witch proteins in the host cells.

In certain embodiments, the Nanchung protein sequences may be selected from the group consisting of a) a polypeptide comprising an amino acid sequence having a sequence selected from the group consisting of SEQ ID NOS: 64-90; b) a polypeptide having at least 70% sequence identity to the SEQ ID NOS: 64-90; and c) a fragment or a conservative variant of the polypeptide of a) or b).

In certain embodiments, the Water witch protein sequences may be selected from the group consisting of a) a polypeptide comprising an amino acid sequence having a sequence selected from the group consisting of SEQ ID NOS: 91-126; b) a polypeptide having at least 70% sequence identity to the SEQ ID NOS: 91-126; and c) a fragment or a conservative variant of the polypeptide of a) or b).

As used herein, the “percent sequence identity” is determined by comparing two optimally aligned sequences over a comparison window, where the portion of the polynucleotide or a polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, divided by the number of matched positions by the total number of positions in the window of comparison and multiplying the result by 100 to yield the percentage of sequence identity. Alignment for the purposes of determining the percentage identity can be achieved in various ways that are within the skill in the art, for example, using publicly available computer software such as BLAST. Those skilled in the art can determine appropriate parameters for measuring alignment, including any algorithms needed to achieve optimal alignment over the full length of the sequences being compared.

As used herein, the term a “conservative variant” refers to an amino acid sequence in which a first amino acid is replaced by a second amino acid or amino acid analog having at least one similar biochemical property, which can be, for example, similar size, charge, hydrophobicity or hydrogen bonding capacity. For example, a first hydrophobic amino acid can be conservatively substituted with a second (non-identical) hydrophobic amino acid such as alanine, valine, leucine, or isoleucine, or an analog thereof. Similarly, a first basic amino acid can be conservatively substituted with a second (non-identical) basic amino acid such as arginine or lysine, or an analog thereof. In the same way, a first acidic amino acid can be conservatively substituted with a second (non-identical) acidic amino acid such as aspartic acid or glutamic acid, or an analog thereof or an aromatic amino acid such as phenylalanine can be conservatively substituted with a second aromatic amino acid or amino acid analog, for example tyrosine. In some embodiments, the peptide comprises conservative variant substitution of at least one amino acid, e.g. 1, 2, 3, 4, 5, 6, 7, 8, 9, 10 or more amino acids. Typically, a conservative variant will retain at least 50%, 55%, 60%, 65%, 70%, 75%, 80%, 85%, 90%, 95%, 97%, 98%, 99% or more of the activity of the wild-type peptide sequence.

Exemplary conservative variant substitution include, but are not limited to, replacement of Alanine (A) with D-ala, Gly, Aib, β-Ala, Acp, L-Cys, or D-Cys; Arginine (R) with D-Arg, Lys, D-Lys, homo-Arg, D-homo-Arg, Met, Be, D-Met, or D-Ile; Asparagine (N) with D-Asn, Asp, D-Asp, Glu, D-Glu, Gln, or D-Gln; Aspartic acid (D) with D-Asp, D-Asn, Asn, Glu, D-Glu, Gln, or D-Gln; Cysteine (C) with D-Cys, S-Me-Cys, Met, D-Met, Thr, or D-Thr; Glutamine (Q) with D-Gln, Asn, D-Asn, Glu, D-Glu, Asp, or D-Asp; Glutamic Acid (E) with D-Glu, D-Asp, Asp, Asn, D-Asn, Gln, or D-Gln; Glycine (G) with Ala, D-Ala, Pro, D-Pro, Aib, β-Ala, or Acp; Isoleucine (I) with D-Ile, Val, D-Val, AdaA, AdaG, Leu, D-Leu, Met, or D-Met; Leucine (L) with D-Leu, Val, D-Val, AdaA, AdaG, Leu, D-Leu, Met, or D-Met; Lysine (K) with D-Lys, Arg, D-Arg, homo-Arg, D-homo-Arg, Met, D-Met, Ile, D-Ile, Orn, or D-Orn; Methionine (M) with D-Met, S-Me-Cys, Be, D-Ile, Leu, D-Leu, Val, or D-Val; Phenylalanine (F) with D-Phe, Tyr, D-Thr, L-Dopa, His, D-His, Trp, D-Trp, Trans-3,4 or 5-phenylproline, AdaA, AdaG, cis-3, 4 or 5-phenylproline, Bpa, or D-Bpa; Proline (P) with D-Pro, L-I-thioazolidine-4-carboxylic acid, or D- or -L-1-oxazolidine-4-carboxylic acid (U.S. Pat. No. 4,511,390); Serine (S) with D-Ser, Thr, D-Thr, allo-Thr, Met, D-Met, Met (O), D-Met (O), L-Cys, or D-Cys; Threonine (T) with D-Thr, Ser, D-Ser, allo-Thr, Met, D-Met, Met (O), D-Met (O), Val, or D-Val; Tyrosine (Y) with D-Tyr, Phe, D-Phe, L-Dopa, His, or D-His; and Valine (V) with D-Val, Leu, D-Leu, Ile, D-Ile, Met, D-Met, AdaA, or AdaG.

Conservative variants of the insect or mammalian TRPV and/or TRPA channels can be prepared according to methods for altering peptide sequences known in the art, and include those that may be found in references, which compile such methods, e.g., Molecular Cloning: A Laboratory Manual, J. Sambrook, et al., eds., Second Edition, Cold Spring Harbor, or Current Protocols in Molecular Biology, F. M. Ausubel, et al., eds., John Wiley & Sons, Inc. New York.

Conservative variants of TRPV and/or TRPA channel may also be made by alteration of a nucleic acid encoding the TRPV and/or TRPA polypeptide.

In certain embodiments, the screening method of the present invention may be a high-throughput screening. High-throughput screening (HTS) refers to a method for scientific experimentation that uses robotics, data processing and control software, liquid handling devices, and sensitive detectors. High-Throughput Screening or HTS allows a researcher to quickly conduct millions of biochemical, genetic or pharmacological tests. HTS is well known in the art, including, for example, U.S. Pat. Nos. 5,976,813; 6,472,144; 6,692,856; 6,824,982; and 7,091,048.

HTS uses automation to run a screen of an assay against a library of candidate compounds. An assay is a test for specific activity: usually inhibition or stimulation of a biochemical or biological mechanism. Specifically, an assay would be screening for inhibition or stimulation of the insect TRPV channel. Typical HTS screening libraries or “decks” can contain from 100,000 to more than 2,000,000 compounds.

The key labware or testing vessel of HTS is the microtiter plate, which is a small container, usually disposable and made of plastic that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, a researcher fills each well of the plate with the appropriate reagents that he or she wishes to conduct the experiment with. After some incubation time has passed to allow the reagent to absorb, bind to, or otherwise react (or fail to react) with the compounds in the wells, measurements are taken across all the plate’s wells, either manually or by a machine. Manual measurements are often necessary when the researcher is using microscopy to (for example) seek changes that a computer could not easily determine by itself. Otherwise, a specialized automated analysis machine can run a number of experiments on the wells such as current or voltage measurements, colorimetric measurements, radioactivity counting, etc. In this case, the machine outputs the result of each experiment as a grid of numeric values, with each number mapping to the value obtained from a single well. A high-capacity analysis machine can measure dozens of plates in the space of a few minutes like this, generating thousands of experimental data points very quickly.

### IV. Formulations, Applications and Uses

In another aspect, the invention provides a compound selected by the screening assay described herein. It is to be understood that analogs, derivatives, isomers, and pharmaceutically acceptable salts of the compounds selected by the screening assays described herein as well as any formulations including the selected compound are also included herein.

The compound or group of compounds being determined or identified by the method according to the invention can be used in methods of insect control, e.g., by modulating insect feeding behavior. The identified compounds, analogs, derivatives, isomers, and pharmaceutically acceptable salts thereof are also referred to as “active agents” or “active ingredients” herein.

Without being bound by theory, the modulation of an insect TRPV and/or TRPA channels elicits a signaling pathway which may kill insect, perturb insect behavior or repel insect. Thus, in some embodiments, the method comprises modulation of TRPV ion channel and/or TRPA channel with a compound identified by a screening method described herein.

Accordingly, in certain embodiments, the invention provides a method of insect control by killing insects, perturbing insect behaviour or repelling an insect using a compound identified by the screening methods described herein. As used in context of methods of insect control, compounds identified by the screening methods described herein also include analogs, derivatives, isomers and pharmaceutically acceptable salts of such compounds.

In one embodiment, the methods and active agents described herein are applicable to insects that are agricultural or horticultural pest.

Examples of agricultural pests include, but are not limited to,Aphids (including Aphis fabae, Aphis gossypii, Aphis pomi, Aulacorthum solani, Brevicoryne brassicae, Dysaphis plantaginea, Macrosiphum euphorbiae, Macrosiphum euphorbiae, Macrosiphum rosae, Myzus persicae, Nasonovia ribisnigri); Whiteflies (including Aleurodes spp., Bemisia spp., Dialeurodes spp., Trialeurodes spp.); Planthoppers (including Laodelphax striatellus, Nilaparvata lugens, Siphanta spp., Sogatella furcifera); Leafhoppers (including Amrasca spp., Empoasca spp.); Scales (including Aspidiotus spp., Chrysomphalus aonidum, Icerya purchase, Unaspis citri); Mealybugs (including Maconellicoccus spp., Paracoccus spp., Planococcus spp.); Pollen beetles (including Carpophilus spp., Meligethes spp.); Thrips (including Caliothrips spp., Frankliniella spp., Scirtothrips spp., Thrips spp.); and Psyllids (including Bactericera spp., Cacopsylla spp., Diaphorina citri, Paratrioza cockerelli).

For example, vegetable and cole crops are sensitive to infestation by one or more of the following insect pests: aphids, brown plant hopper, alfalfa looper, armyworm, beet armyworm, artichoke plume moth, cabbage budworm, cabbage looper, cabbage webworm, corn earworm, celery leafeater, cross-striped cabbagewon, european corn borer, diamondback moth, green cloverworm, imported cabbageworm, melonworm, omnivorous leafroller, pickleworm, rindworm complex, saltmarsh caterpillar, soybean looper, tobacco budworm, tomato fruitworm, tomato homworrri, tomato pinworm, velvetbean caterpillar, and yellow-striped annyworm.

Likewise, pasture and hay crops such as alfalfa, pasture grasses and silage are often attacked by pests, such as annyworm, beef annyworm, alfalfa caterpillar, European skipper, a variety of loopers and webworms, as well as yellowstriped armyworms.

Fruit and vine crops are often susceptible to attack and defoliation by achema sphinx moth, amorbia, armyworm, citrus cutworm, blackheaded fireworm, blueberry leafroller, cankerworm, cherry fruitworm, citrus cutworm, cranberry girdler, eastern tent caterpillar, fall webworm, fall webworm, filbert leafroller, filbert webworm, fruit tree leafroller, grape berry moth, grape leaffolder, grapeleaf skeletonizer, green fruitworm, gummosos-batrachedra commosae, gypsy moth, hickory shuckworm, hornworms, loopers, navel orangeworm, obliquebanded leafroller, orrinivorous leafroller, omnivorous looper, orange tortrix, orangedog, oriental fruit moth, pandemis leafroller, peach twig borer, pecan nut casebearer, redbanded leafroller, redhumped caterpillar, roughskinned cutworm, saltmarsh caterpillar, spanworm, tent caterpillar, thecla-thecla basillides, tobacco budworm, tortrix moth, tufted apple budmoth, variegated leafroller, walnut caterpillar, western tent caterpillar, and yellowstriped annyworm.

Field crops such as canola/rape seed, evening primrose, meadow foam, corn (field, sweet, popcorn), cotton, hops, jojoba, peanuts, rice, safflower, small grains (barley, oats, rye, wheat, etc.), sorghum, soybeans, sunflowers, and tobacco are often targets for infestation by insects, including: armyworm, asian and other corn borers, banded sunflower moth, beet annyworm, bollworm, cabbage looper, corn rootworm (including southern and western varieties), cotton leaf perforator, diamondback moth, european corn borer, green cloverworm, headmoth, headworm, imported cabbagewonn, loopers (including Anacamptodes spp.), obliquebanded leafroller, omnivorous leaftier, podworin, podworm, saltmarsh caterpillar, southwestern corn borer, soybean looper, spotted cutworm, sunflower moth, tobacco budworm, tobacco hornworm, velvetbean caterpillar. Bedding plants, flowers, ornamentals, vegetables and container stock are frequently fed upon by a host of insect pests such as armyworm, azalea moth, beet armyworm, diamondback moth, ello moth (hornworm). Florida fern caterpillar, oleander moth, omnivorous leafroller, omnivorous looper, and tobacco. Forests, fruit, ornamental, and nut-bearing trees, as well as shrubs and other nursery stock are often susceptible to attack from diverse insects such as bagworm, blackheaded budworm, browntail moth, california oakworm, douglas fir tussock moth, elm spanworm, fall webworm, fruit tree leafroller, greenstriped mapleworm, gypsy moth. Jack pine budworm, mimosa webworm, pine butterfly, redhumped caterpillar, saddleback caterpillar, saddle prominent caterpillar, spring and fall cankerworm, spruce budworm, tent caterpillar, tortrix, and western tussock moth.

Likewise, turf grasses are often attacked by pests such as armyworm, sod webworm, and tropical sod webworm.

It is also envisioned that the methods described herein are also applicable to pest control, wherein the pests are not insects but rather, e.g., nematodes, slugs or snails.

In one embodiment, the methods described herein are also applicable to insects that are parasites. Examples of some insect parasites are Braconid Wasps, family Braconidae; Ichneumonid Wasps, family Ichneumonidae; Chalcid Wasps, family Chalcidae; Tachinid Flies, family Tachonidae.

In certain other embodiments, the methods described herein may also be applicable to insects that are disease vectors. Vectors are organisms that can introduce a pathogen such as a bacterium or virus into a host organism to cause an infection or disease. Exemplary disease vector include, but are not limited to, mosquitoes, Ticks, Siphonaptera (fleas), Diptera (flies), Phthiraptera (lice) and Hemiptera (true bugs).

Once a compound suitable for insecticidal use is identified, the active ingredient, or formulations comprising them, may be applied directly to the target insects (i.e., larvae, pupae and/or adults), or to the locus of the insects. In one embodiment, the active ingredient or a formulation and/or composition containing the active ingredient is applied directly to the adult insect. In one embodiment, the active agent is applied directly to the larvae and/or pupae of the target insect. In another embodiment, the active ingredient is applied to the locus of the insects.

Because compounds incorporating hydrophobic moieties will penetrate the insect cuticle, active agents can be conjugated with hydrophobic moieties. Hydrophobic moieties include, but are not limited to, lipids and sterols.

In one embodiment, the active ingredient or a formulation including the active ingredient may be applied as a spray. For example, the active ingredient may be applied as an agricultural spray in aerial crop dusting, an environmental spray to control biting insects, or as a topical spray for localized control of biting insects. The active ingredient may be formulated for the purpose for spray application such as an aerosol formulation. Spray application may be accomplished with a spray pump. The active ingredient may be also encapsulated within materials such as starch, flour and gluten in granular formulations.

In certain embodiments, the active ingredient or a formulation including the active ingredient may be applied in conjunction with other insecticides and/or pesticides such as organo-phosphates, synthetic pyrethroids, carbamates, chlorinated hydrocarbons, when used in agricultural and/or environmental insect control.

The active ingredient may be administered in an amount effective to induce the desired response as determined by routine testing. The actual effective amount will of course vary with the specific active ingredient, the target insect and its stage of development, the application technique, the desired effect, and the duration of the effect, and may be readily determined by the practitioner skilled in the art. “An effective amount of active ingredient” refers to the amount of active ingredient that modulates (activates or inhibits) an insect TRPV channel and/or TRPA channel, e.g., kills insect, perturbs insect behavior or repel insect to achieve the desired insect control.

Methods of formulation are well known to one skilled in the art and are also found in Knowles, D A (1998) Chemistry and technology of agricultural formulations. Kluwer Academic, London, which is hereby incorporated by reference in its entirety. One skilled in the art will, of course, recognize that the formulation and mode of application may affect the activity of the active ingredient in a given application. Thus, for agricultural and/or horticultural use the TRPV inhibitors and/or agonists may be formulated as a granular of relatively large particle size (for example, 8/16 or 4/8 US Mesh), as water-soluble or water-dispersible granules, as powdery dusts, as wettable powders, as emulsifiable concentrates, as aqueous emulsions, as solutions, as suspension concentrate, as capsule suspensions, as soluble (liquid) concentrates, as soluble powders, or as any of other known types of agriculturally-useful formulations, depending on the desired mode of application. It is to be understood that the amounts specified in this specification are intended to be approximate only, as if the word “about” were placed in front of the amounts specified.

These formulations may be applied either as water-diluted sprays, or dusts, or granules in the areas in which insect control is desired. The formulations may contain as little as 0.1%, 0.2% or 0.5% to as much as 95% or more by weight of active ingredient, e.g. insect TRPV inhibitor.

“Dusts” are free flowing admixtures of the active ingredient with finely divided solids such as talc, natural clays, kieselguhr, flours such as walnut shell and cottonseed flours, and other organic and inorganic solids which act as dispersants and carriers for the toxicant; these finely divided solids have an average particle size of less than about 50 microns. A typical dust formulation useful herein is one containing 90 parts, 80 parts, 70 parts, 60 parts, 50 parts, 40 parts, 30 parts, 20 parts, preferably 10 parts, or less of the active ingredient, e.g., insect TRPV modulator or insect TRPA modulator. In one embodiment, the dust formulation may include 1 part or less of the active ingredient and 99 parts or more of talc.

Wettable powders, useful as formulations, are in the form of finely divided particles that disperse readily in water or another dispersant. The wettable powder is ultimately applied to the locus where insect control is needed either as a dry dust or as an emulsion in water or other liquid. Typical carriers for wettable powders include Fuller’s earth, kaolin clays, silicas, and other highly absorbent, readily wet inorganic diluents. Wettable powders typically are prepared to contain about 5-80% of active ingredient, depending on the absorbency of the carrier, and usually also contain a small amount of a wetting, dispersing or emulsifying agent to facilitate dispersion. For example, a useful wettable powder formulation contains 80.0 parts of the active ingredient, 17.9 parts of Palmetto clay, and 1.0 part of sodium lignosulfonate and 0.3 part of sulfonated aliphatic polyester as wetting agents. Additional wetting agent and/or oil may be added to a tank mix for to facilitate dispersion on the foliage of the plant.

Other useful formulations are emulsifiable concentrates (ECs) which are homogeneous liquid compositions dispersible in water or other dispersant, and may consist entirely of the active ingredient, and a liquid or solid emulsifying agent, or may also contain a liquid carrier, such as xylene, heavy aromatic naphthas, isophorone, or other non-volatile organic solvents. For insecticidal application these concentrates are dispersed in water or other liquid carrier and normally applied as a spray to the area to be treated. The percentage by weight of the essential active ingredient may vary according to the manner in which the composition is to be applied, but in general comprises 0.5 to 95% of active ingredient by weight of the insecticidal composition.

Flowable formulations are similar to ECs, except that the active ingredient is suspended in a liquid carrier, generally water. Flowables, like ECs, may include a small amount of a surfactant, and will typically contain active ingredients in the range of 0.5 to 95%, frequently from 10 to 50%, by weight of the composition. For insecticidal application, flowables may be diluted in water or other liquid vehicle, and are typically applied as a spray.

Typical wetting, dispersing or emulsifying agents used in agricultural and/or horticultural formulations may include, but are not limited to, the alkyl and alkylaryl sulfonates and sulfates and their sodium salts; alkylaryl polyether alcohols; sulfated higher alcohols; polyethylene oxides; sulfonated animal and vegetable oils; sulfonated petroleum oils; fatty acid esters of polyhydric alcohols and the ethylene oxide addition products of such esters; and the addition product of long-chain mercaptans and ethylene oxide. Many other types of useful surface-active agents are available in commerce. Surface-active agents, when used, typically include 1 to 15% by weight of the composition.

Other useful formulations include suspensions of the active ingredient in a relatively non-volatile solvent such as water, corn oil, kerosene, propylene glycol, or other suitable solvents.

In certain embodiments, formulations for insecticidal applications may include simple solutions of the active ingredient in a solvent, in which it is completely soluble at the desired concentration, such as acetone, alkylated naphthalenes, xylene, or other organic solvents.

Granular formulations, wherein the active ingredient is carried on relative coarse particles, are of particular utility for aerial distribution or for penetration of cover crop canopy. Pressurized sprays, typically aerosols wherein the active ingredient is dispersed in finely divided form as a result of vaporization of a low-boiling dispersant solvent carrier may also be used. Water-soluble or water-dispersible granules are free flowing, non-dusty, and readily water-soluble or water-miscible. In use by the farmer on the field, the granular formulations, emulsifiable concentrates, flowable concentrates, aqueous emulsions, solutions, etc., may be diluted with water to give a concentration of active ingredient in the range of say 0.1% or 0.2% to 1.5% or 2%.

By far the most frequently used are water-miscible formulations for mixing with water then applying as sprays. Water miscible, older formulations include: emulsifiable concentrate, wettable powder, soluble (liquid) concentrate, and soluble powder. Newer, non-powdery formulations with reduced or no hazardous solvents and improved stability include: suspension concentrate, capsule suspensions, and water dispersible granules. Such formulations are preferably solutions and suspension, e.g., aqueous suspension and solutions, ethanolic suspension and solutions, aqueous/ethanolic suspension and solutions, saline solutions, and colloidal suspensions.

Alternatively, a sprayable wax emulsion formulation may be used. The formulation contains the active ingredient, in an amount from about 0.01% to 75% by weight. The aqueous wax emulsions are broadly described in U.S. Pat. No. 6,001,346, which is hereby incorporated by reference in its entirety. The TRPV and/or TRPA modulators of the methods described herein can have a viscosity appropriate for use in aerial or backpack spray applications.

The biodegradable wax carrier comprises at least about 10% by weight of the formulation. The biodegradable wax carrier is selected from the group consisting of paraffin, beeswax, vegetable based waxes such as soywax (soybean based), and hydrocarbon based waxes such as Gulf Wax Household Paraffin Wax; paraffin wax, avg. m.p. 53C (hexacosane), high molecular weight hydrocarbons), carnauba wax, lanolin, shellac wax, bayberry wax, sugar cane wax, microcrystalline, ozocerite, ceresin, montan, candelilla wax, and combinations thereof.

Formulations may also contain an emulsifier in an amount from about 1% to about 10% by weight. Suitable emulsifiers include lecithin and modified lecithins, mono- and diglycerides, sorbitan monopalmitate, sorbitan monooleate, sorbitan monolaurate, polyoxyethylene-sorbitan monooleate, fatty acids, lipids, etc. The emulsifiers provide or improve emulsification properties of the composition. The emulsifier can be selected from many products which are well known in the art, including, but not limited to, sorbitan monolaurate (anhydrosorbitol stearate, molecular formula C₂₄H₄₆O₆), ARLACEL 60, ARMOTAN MS, CRILL 3, CRILL K3, DREWSORB 60, DURTAN 60, EMSORB 2505, GLYCOMUL S, HODAG SMS, IONET S 60, LIPOSORB S, LIPOSORB S-20, MONTANE 60, MS 33, MS33F, NEWCOL 60, NIKKOL SS 30, NISSAN NONION SP 60, NONION SP 60, NONION SP 60R, RIKEMAL S 250, sorbitan c, sorbitan stearate, SORBON 60, SORGEN 50, SPAN 55, AND SPAN 60; other sorbitan fatty acid ester that may be used include sorbitan monopalmitate, sorbitan monostearate, sorbitan tristearate, sorbitan monooleate, sorbitan sesquioleate, sorbitan trioleate, sorbitan monooleate, sorbitan trioleate.

In certain embodiments, formulations can include a phagostimulant, such as corn oil, molasses, glycerol, or corn syrup, proteinaceous material (protein or hydrolyzed protein), sugars like sucrose, or food-based ingredients such as trimethylamine, putrescine, bacterial or yeast volatiles or metabolites, ammonium acetate, ammonium carbonate or other ammonia-emitting compounds. Acetic acid vapor can be provided by compounds that produce volatilized acetic acid, for example, aqueous acetic acid, glacial acetic acid, glacial (concentrated) acetic acid, or ammonium producing compounds such as but not restricted to ammonium hydroxide, ammonium carbonate, ammonium bicarbonate, ammonium acetate, etc.

In certain embodiments, the active ingredient may be formulated and/or applied with one or more second compounds. For example, various combinations of TRPV modulators and /or TRPA modulators may be used to obtain greater advantage. For example, both a TRPV modulator and TRPAmodulator may be applied at the same time. As such in one embodiment, a formulation described herein may include both, a TRPV modulator and a TRPA modulator. In one embodiment, two or more active agents may be formulated together. In alternative embodiment, two or more active agents formulated together are all either TRPA modulators or are all TRPV modulators. Such combinations may provide certain advantages, such as, without limitation, exhibiting synergistic effects for greater control of insects or non-insect pests, reducing rates of application thereby minimizing any impact to the environment and to worker safety, controlling a broader spectrum of insects and non-insect pests, and improving tolerance by non-pest species, such as mammals and fish. Other second compounds include, without limitation, insecticides, pesticides, plant growth regulators, fertilizers, soil conditioners, or other agricultural and horticultural chemicals. The formulation may include such second compounds in an amount from about 0.002% to about 25% by weight of the composition.

In certain embodiments, the formulations of the present invention may contain visual attractants, e.g. food coloring.

A variety of additives may also be incorporated into the formulation. These additives typically change and/or enhance the physical characteristics of the carrier material and are, therefore, suitable for designing compositions having specific requirements as to the release rate and amount of the active ingredient, protection of the wax composition from weather conditions, etc. These additives are, among others, plasticizers, volatility suppressants, antioxidants, lipids, various ultraviolet blockers and absorbers, or antimicrobials, typically added in amounts from about 0.001% to about 10%, more typically between 1-6%, by weight.

Plasticizers, such as glycerin or soy oil affect physical properties of the composition and may extend its resistance to environmental destruction.

Antioxidants, such as vitamin E, BHA (butylated hydroxyanisole), BHT (butylated hydroxytoluene), and other antioxidants which protect the bioactive agent from degradation, may be added in amounts from about 0.1% to about 3%, by weight.

Ultraviolet blockers, such as beta-carotene, lignin or p-aminobenzoic acid protect the bioactive agents from light degradation may be added in amounts from about 1% to about 3%, by weight.

Antimicrobials, such as potassium sorbate, nitrates, nitrites, and propylene oxide, protect the bioactive agents from microbial destruction may be added in amounts from 0.1% to about 2% by weight.

Adjuvants can also be added to the formulation. An “adjuvant” is broadly defined as any substance added to the spray tank, separate from the insecticide formulation that will improve the performance of the insecticide. These may include, but are not limited to: wetter-spreaders, stickers, penetrants, compatibility agents, buffers, and so on.

Other compounds and materials may also be added provided they do not substantially interfere with the activity of active ingredient. Whether or not an additive substantially interferes with the active ingredient’s activity can be determined by standard test formats, involving direct comparisons of efficacy of the composition of the active ingredient without an added compound and the composition of the active ingredient with an added compound.

It was discovered that an insect will stop eating after ingesting a TRPV channel activating compound. For example, ingesting a TRPV and/or TRPA ion channel agonist can cause an insect to stop eating. Thus, in one embodiment, the compounds may be formulated with a food source for insects, e.g., formulated with compounds in insect diet. In another embodiment, the compounds may be formulated with sucrose. Without wishing to be bound by theory, the insect will feed on such mixtures and stop eating.

In certain embodiments, the active agent may be applied to feeding locus of insects. This inhibits insect feeding leading to starvation of insects. In one embodiment, the active agent is applied as a spray to locus of insects, e.g., feeding locus. The active agent may be applied to the insect and/or pest (mollusk, nematode and/or arthropod) feeding source, used in bait or traps, and or directly applied to the pest itself.

In one embodiment, the active agent may be applied to insect traps. For example, the trap may be coated with the active agent or trap may be loaded with insect food comprising an active agent.

The nucleic acid sequences of Nanchung and Water witch subunits of the TRP channels as well as the corresponding amino acid sequences, their origins, accession numbers (if available) and host organism codes are summarized in Table 1 below. Upon co-expression of the Nanchung and Water witch proteins in a cell, a functional TRP channel may be formed.

TABLE 1

| SEQ ID NO. | DESCRIPTION | ORGANISM | ACCESSION NO. | CODE |
|---|---|---|---|---|
| 1 | Nanchung DNA | Aedes aegypti | XM_001652374.2 | Aaeg |
| 2 | Nanchung DNA | Culex quinquefasciatus | XM_001847084.1 | Cqui |
| 3 | Nanchung DNA | Anopheles darlingi | In-house | Adar |
| 4 | Nanchung DNA | Anopheles gambiae | XM_320300.4 | Agam |
| 5 | Nanchung DNA | Ceratitis capitata | XM_004537685.2 | Ccap |
| 6 | Nanchung DNA | Musca domestica | XM_005180432 | Mdom |
| 7 | Nanchung DNA | Drosophila melanogaster | NM_140439.3 | Dmel |
| 8 | Nanchung DNA | Acromyrmex echinatior | In-house | Aech |
| 9 | Nanchung DNA | Solenopsis invicta | XM_011167328.2 | Sinv |
| 10 | Nanchung DNA | Camponotus floridanus | GL440604.1 | Cflo |
| 11 | Nanchung DNA | Harpegnathos saltator | GL450384.1 | Hsal |
| 12 | Nanchung DNA | Apis florea | XM_003689958.2 | Aflo |
| 13 | Nanchung DNA | Apis mellifera | XM_625167.6 | Amel |
| 14 | Nanchung DNA | Megachile rotundata | XM_003706124.2 | Mrot |
| 15 | Nanchung DNA | Nasonia vitripennis | XM_001606052.4 | Nvit |
| 16 | Nanchung DNA | Dendroctonus ponderosae | In-house | Dpon |
| 17 | Nanchung DNA | Tribolium castaneum | XM_962803.3 | Tcas |
| 18 | Nanchung DNA | Nilaparvata lugens | MF684871.1 | Nlug |
| 19 | Nanchung DNA | Acyrthosiphon pisum | XM_016802803.1 | Apis |
| 20 | Nanchung DNA | Myzus persicae | XM_022324691.1 | Mper |
| 21 | Nanchung DNA | Bemisia tabaci | XM_019045280.1 | Btab |
| 22 | Nanchung DNA | Halyomorpha halys | XM_014421401.2 | Hhal |
| 23 | Nanchung DNA | Euschistus heros | In-house | Eher |
| 24 | Nanchung DNA | Pediculus humanus corporis | XM_002427918.1 | Phum |
| 25 | Nanchung DNA | Bombyx mori | XM_021353316.1 | Bmor |
| 26 | Nanchung DNA | Vanessa tameamea | XM_026628835.1 | Vtam |
| 27 | Nanchung DNA | Danaus plexippus | In-house | Dple |
| 28 | Water witch DNA | Acromyrmex echinatior | In-house | Aech |
| 29 | Water witch DNA | Apis florea | XM_003691355.2 | Aflo |
| 30 | Water witch DNA | Apis mellifera | XM_026439154.1 | Amel |
| 31 | Water witch DNA | Bombus terrestris | XM_012315059.2 | Bter |
| 32 | Water witch DNA | Nasonia vitripennis | XM_008212470.2 | Nvit |
| 33 | Water witch DNA | Cimex lectularius | XM_014387113.2 | Clec |
| 34 | Water witch DNA | Halyomorpha halys | XM_014427168.2 | Hhal |
| 35 | Water witch DNA | Nezara viridula | In-house | Nvir |
| 36 | Water witch DNA | Nilaparvata lugens | XM_022345460.1 | Nlug |
| 37 | Water witch DNA | Acyrthosiphon pisum | XM_016802413.1 | Apis |
| 38 | Water witch DNA | Myzus persicae | XM_022315379.1 | Mper |
| 39 | Water witch DNA | Diaphorina citri | XM_026824678.1 | Dcit |
| 40 | Water witch DNA | Pediculus humanus corporis | XM_002430593.1 | Phum |
| 41 | Water witch DNA | Dendroctonus ponderosae | XM_019911745.1 | Dpon |
| 42 | Water witch DNA | Leptinotarsa decemlineata | XM_023164353.1 | Ldec |
| 43 | Water witch DNA | Tribolium castaneum | XM_961536.3 | Tcas |
| 44 | Water witch DNA | Plutella xylostella | XM_011556111.1 | Pxyl |
| 45 | Water witch DNA | Trichoplusia ni | XM_026879699.1 | Trni |
| 46 | Water witch DNA | Amyelois transitella | XM_013330567.1 | Atra |
| 47 | Water witch DNA | Helicoverpa armigera | XM_021338514.1 | Harm |
| 48 | Water witch DNA | Spodoptera littoralis | XM_022959442.1 | Slit |
| 49 | Water witch DNA | Papilio xuthus | XM_013310920.1 | Pxut |
| 50 | Water witch DNA | Papilio machaon | XM_014508830.1 | Pmac |
| 51 | Water witch DNA | Vanessa tameamea | XM_026627409.1 | Vtam |
| 52 | Water witch DNA | Bombyx mori | NM_001309607.1 | Bmor |
| 53 | Water witch DNA | Anopheles gambiae | XM_319460.3 | Agam2 |
| 54 | Water witch DNA | Aedes aegypti | XM_021853484.1 | Aaeg |
| 55 | Water witch DNA | Culex quinquefasciatus | XM_001851266.1 | Cqui |
| 56 | Water witch DNA | Anopheles gambiae | XM_310750.5 | Agam1 |
| 57 | Water witch DNA | Lucilia cuprina | XM_023444358.1 | Lcup |
| 58 | Water witch DNA | Musca domestica | XM_005183066.3 | Mdom |
| 59 | Water witch DNA | Stomoxys calcitrans | XM_013253516.1 | Scal |
| 60 | Water witch DNA | Bactrocera dorsalis | XM_011200137.2 | Bdor |
| 61 | Water witch DNA | Zeugodacus cucurbitae | XM_011186538.1 | Zcuc |
| 62 | Water witch DNA | Ceratitis capitata | XM_012303232.2 | Ccap |
| 63 | Water witch DNA | Drosophila melanogaster | NM_169200.2 | Dmel |
| 64 | Nanchung PROT | Aedes aegypti | XP_001652424.1 | Aaeg |
| 65 | Nanchung PROT | Culex quinquefasciatus | XP_001847136.1 | Cqui |
| 66 | Nanchung PROT | Anopheles darlingi | In-house | Adar |
| 67 | Nanchung PROT | Anopheles gambiae | XP_320300.4 | Agam |
| 68 | Nanchung PROT | Ceratitis capitata | XP_004537742.1 | Ccap |
| 69 | Nanchung PROT | Musca domestica | XP_005180489.1 | Mdom |
| 70 | Nanchung PROT | Drosophila melanogaster | NP_648696.2 | Dmel |
| 71 | Nanchung PROT | Acromyrmex echinatior | In-house | Aech |
| 72 | Nanchung PROT | Solenopsis invicta | XP_011165630.1 | Sinv |
| 73 | Nanchung PROT | Camponotus floridanus | EFN65752.1 | Cflo |
| 74 | Nanchung PROT | Harpegnathos saltator | EFN81068.1 | Hsal |
| 75 | Nanchung PROT | Apis florea | XP_003690006.1 | Aflo |
| 76 | Nanchung PROT | Apis mellifera | XP_625170.3 | Amel |
| 77 | Nanchung PROT | Megachile rotundata | XP_003706172.1 | Mrot |
| 78 | Nanchung PROT | Nasonia vitripennis | XP_001606102.2 | Nvit |
| 79 | Nanchung PROT | Dendroctonus ponderosae | In-house | Dpon |
| 80 | Nanchung PROT | Tribolium castaneum | XP_967896.1 | Tcas |
| 81 | Nanchung PROT | Nilaparvata lugens | ATU07272.1 | Nlug |
| 82 | Nanchung PROT | Acyrthosiphon pisum | XP_016658292.1 | Apis |
| 83 | Nanchung PROT | Myzus persicae | XP_022180383.1 | Mper |
| 84 | Nanchung PROT | Bemisia tabaci | XP_018900825.1 | Btab |
| 85 | Nanchung PROT | Halyomorpha halys | XP_014276887.1 | Hhal |
| 86 | Nanchung PROT | Euschistus heros | In-house | Eher |
| 87 | Nanchung PROT | Pediculus humanus corporis | XP_002427963.1 | Phum |
| 88 | Nanchung PROT | Bombyx mori | XP_021208991.1 | Bmor |
| 89 | Nanchung PROT | Vanessa tameamea | XP_026484620.1 | Vtam |
| 90 | Nanchung PROT | Danaus plexippus | In-house | Dple |
| 91 | Water witch PROT | Acromyrmex echinatior | In-house | Aech |
| 92 | Water witch PROT | Apis florea | XP_003691403.1 | Aflo |
| 93 | Water witch PROT | Apis mellifera | XP_026294939.1 | Amel |
| 94 | Water witch PROT | Bombus terrestris | XP_012170449.1 | Bter |
| 95 | Water witch PROT | Nasonia vitripennis | XP_008210692.1 | Nvit |
| 96 | Water witch PROT | Cimex lectularius | XP_014242599.1 | Clec |
| 97 | Water witch PROT | Halyomorpha halys | XP_014282654.1 | Hhal |
| 98 | Water witch PROT | Nezara viridula | In-house | Nvir |
| 99 | Water witch PROT | Nilaparvata lugens | XP_022201152.1 | Nlug |
| 100 | Water witch PROT | Acyrthosiphon pisum | XP_016657902.1 | Apis |
| 101 | Water witch PROT | Myzus persicae | XP_022171071.1 | Mper |
| 102 | Water witch PROT | Diaphorina citri | XP_026680479.1 | Dcit |
| 103 | Water witch PROT | Pediculus humanus corporis | XP_002430638.1 | Phum |
| 104 | Water witch PROT | Dendroctonus ponderosae | XP_019767304.1 | Dpon |
| 105 | Water witch PROT | Leptinotarsa decemlineata | XP_023020121.1 | Ldec |
| 106 | Water witch PROT | Tribolium castaneum | XP_966629.2 | Tcas |
| 107 | Water witch PROT | Plutella xylostella | XP_011554413.1 | Pxyl |
| 108 | Water witch PROT | Trichoplusia ni | XP_026735500.1 | Trni |
| 109 | Water witch PROT | Amyelois transitella | XP_013186021.1 | Atra |
| 110 | Water witch PROT | Helicoverpa armigera | XP_021194189.1 | Harm |
| 111 | Water witch PROT | Spodoptera littoralis | XP_022815210.1 | Slit |
| 112 | Water witch PROT | Papilio Xuthus | XP_013166374.1 | Pxut |
| 113 | Water witch PROT | Papilio machaon | XP_014364316.1 | Pmac |
| 114 | Water witch PROT | Vanessa tameamea | XP_026483194.1 | Vtam |
| 115 | Water witch PROT | Bombyx mori | NP_001296536.1 | Bmor |
| 116 | Water witch PROT | Anopheles gambiae | XP_319460.3 | Agam2 |
| 117 | Water witch PROT | Aedes aegypti | XP_021709176.1 | Aaeg |
| 118 | Water witch PROT | Culex quinquefasciatus | XP_001851318.1 | Cqui |
| 119 | Water witch PROT | Anopheles gambiae | XP_310750.5 | Agam1 |
| 120 | Water witch PROT | Lucilia cuprina | XP_023300126.1 | Lcup |
| 121 | Water witch PROT | Musca domestica | XP_005183123.1 | Mdom |
| 122 | Water witch PROT | Stomoxys calcitrans | XP_013108970.1 | Scal |
| 123 | Water witch PROT | Bactrocera dorsalis | XP_011198439.1 | Bdor |
| 124 | Water witch PROT | Zeugodacus cucurbitae | XP_011184840.1 | Zcuc |
| 125 | Water witch PROT | Ceratitis capitate | XP_012158622.1 | Ccap |
| 126 | Water witch PROT | Drosophila melanogaster | NP_731193.1 | Dmel |

Note: In-house sequences were generated through public and proprietary sequence curation.

Adenovirions, which include the adenovirus expression vectors of the present invention, can be produced using the following methodology. Adenovirons may be used to generate cells used to express the proteins of interest, for example, Nanchung, and/or Water witch proteins.

The methods of creating an adenovirus generally involves the steps of introducing the vector containing the gene of interest (e.g., Nanchung or Water witch) into a producer cell. The vector containing the gene of interest may be introduced into the producer cell using standard transfection techniques known to one of skill in the art (Zoltukhin et al., Gene Therapy, 6:973-985, 1999). The adenovirus produced by producer cells (packaging cells) are used to transduce a cell of interest. The cell of interest produces the desired proteins.

Preferably, the adenovirions are added to the cells at the appropriate multiplicity of infection according to standard transduction methods appropriate for the particular target cells. Titers of adenovirions to administer can vary, depending upon the target cell type and the particular viral vector, and may be determined by those of skill in the art without undue experimentation.

The cell line may be an insect cell line, such as Sf9 (ATCC# CRL-1711) or Schneider 2 (S2) cells (Life Technologies, #R690-07) (if needed, to introduce a gene of interest into an insect cell transfection techniques may be used among others), African clawed frog (Xenopus laevis) oocytes (to introduce a gene of interest injection techniques may be used), or a mammalian cell line, such as mouse, hamster, human cell line, (to introduce a gene of interest into a mammalian cell several techniques may be used, including but not limited to transfection, adenoviruses, retroviruses, and/or electroporation, etc...) or any other cell line that does not normally expresses Nanchung and Water witch proteins. One example of a mammalian cell line suitable for use with the vector expression system of the present invention includes Chinese hamster ovary (CHO-K1) cells (ATCC# CCL-61).

In some embodiments, a cell expressing a recombinant nucleic acid sequence encoding a TRPV and/or TRPA channel is a cell that has been transformed with an expression vector comprising a nucleotide sequence encoding an insect TRPV and/or TRPA channel such as, but not limited to the TRP channel proteins discloses herein. Methods for transforming cells that would be known to one of ordinary skill in the art include, but are not limited to, infection using viral vectors, lipofection, electroporation, particle bombardment, and transfection. Detailed procedures for representative methods can be found in Sambrook & Russell, 2001 and references cited therein. Useful expression vectors and methods of introducing such vectors into cells or expression of the encoded polypeptide are also known to one of ordinary skill in the art. For example, a plasmid expression vector can be introduced into a cell by calcium-phosphate mediated transfection, DEAE-Dextran-mediated transfection, lipofection, polybrene- or polylysine-mediated transfection, electroporation, or by conjugation to an antibody, gramicidin S, artificial viral envelopes, or other intracellular carriers. A viral expression vector can be introduced into a cell in an expressible form by infection or transduction, for example, or by encapsulation in a liposome.

When a cell expressing a recombinant nucleic acid sequence encoding an insect TRPV and/or TRPA channel gene product has been produced, these cells can then be employed in testing candidate compounds for an ability to modulate ion transport in the cell through the TRPA and/or TRPV channel. The exemplary methods for testing ion transport in the cells were described above as well as presented in the Examples sections below. Other applicable methods would be known to those of skill in the art upon consideration of this disclosure.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example I: Adenovirus Expression Constructs

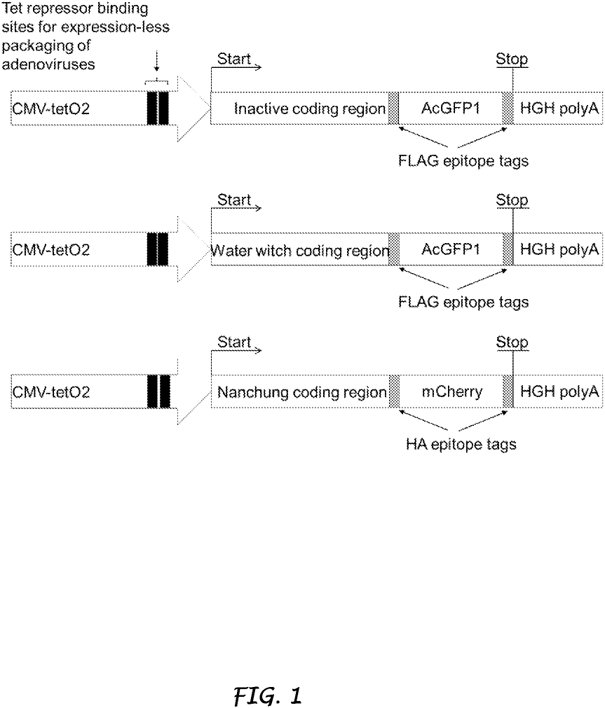

To test biological activity and ligand binding properties of TRP channels, Drosophila melanogaster Nanchung, Inactive and Water witch proteins were expressed in hamster CHO-K1 cells either alone or in combinations. To trace expression of the TRP channels they were fused with fluorescent proteins, AcGFP and mCherry, as well as antibody epitope tags HA and FLAG, as illustrated in FIG. 1. DNAs encoding for tagged TRP channels were delivered into CHO-K1 cells using adenoviruses. Adenovirus-mediated gene delivery allowed for expression of the gene of interest, optimize expression levels and stoichiometry of subunits, as well as prevention of possible toxic effects of insect TRP channels by maintaining virus-infected cells at room temperature.

To prevent toxicity of TRP channels during adenovirus packaging stage, adenoviral constructs containing Tet repressor binding site within modified CMV promoter were used (FIG. 1). The presence of Tet repressor binding sites inhibits expression of the genes of interest in adenovirus packaging cells producing Tet repressor.

### Example II: Activation of TRP Channels by Afidopyropen and Pymetrozine

It has previously been shown that commercial insecticides afidopyropen (AP) and pymetrozine (PM) hyperactivate activate and eventually silence complexes consisting of Nanchung (Nan) and Inactive (Iav). Nanchung and Inactive are present in insect chordotonal organs, mechanosensors involved in insect body coordination, as well as sensing sound and gravity (Kavlie and Albert, 2013). Nanchung was shown to be a direct target of the insecticides, whereas Inactive was required for biological activity and high affinity ligand binding (Kandasamy et al., 2017).

TRPA channel Water witch (Water witch) is also present in chordotonal organs, and point mutation within conservative region of Water witch, which is presumably involved in ion conductivity, produced effects, which resmemble the effects of AP or PM (Senthilan et al., 2012). To test the hypothesis that Water witch may also be involved in the action of PM and AP, Water witch was expressed in CHO-K1 cells either alone or in combination with Nanchung. Given that TRP channels are permeable for Ca2+, cell responces were measured by monitoring Ca2+ mobilization using Ca2+ sensitive fluorescent probe FLUO4.

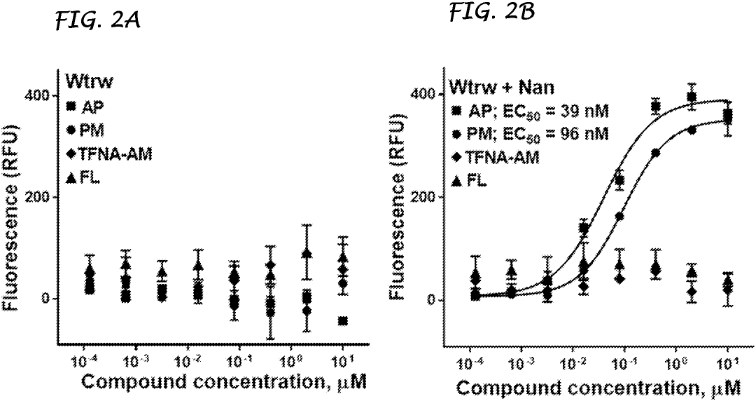

As shown on FIG. 2, both PM and AP elicited calcium mobilization in the cells, co-expressing Nanchung and Water witch (FIG. 2B), but not cells expressing Water witch individually (FIG. 2A). It has been previously shown that Nanchung alone does not respond to stimulation by AP or PM, and required co-expression of Inactive for biological activity. The effect of Water witch co-expression with Nanchung shown on FIG. 2 is, therefore, analogous to the effect of Inactive co-expression with Nanchung.

Notheworthy, two other insecticidal compounds, flonicamid (FL), and TFNA-AM, which also act on chordotonal organs, but do not modulate insect TRPV channels were without effect.

### Example III: Nanchung and Water Witch Assemple Into Nanchung:Water Witch Complexes.

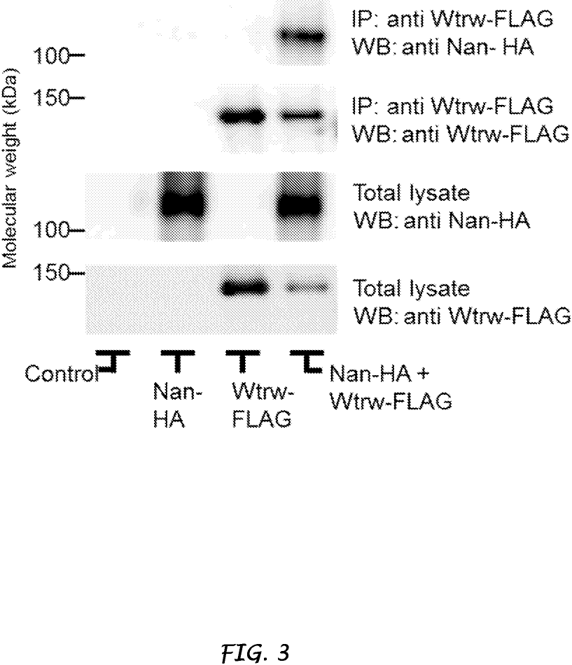

The observation that calcium mobilization elicited by AP and PM required co-expression of Nanchung and Water witch suggested that these two proteins may assemble into Nanchung:Water witch complexes. To test this hypothesis, Nanchung and Water witch were fused with different antibody epitope tags. As depicted by FIG. 3, HA-tagged Nanchung co-immunoprecipitated with FLAG-tagged Water witch, analogous to previously reported association of Nanchung with Inactive.

### Example IV: Nanchung-Water Witch Complexes Are Activated by the Endogenous Agonist, Nicotinamide.

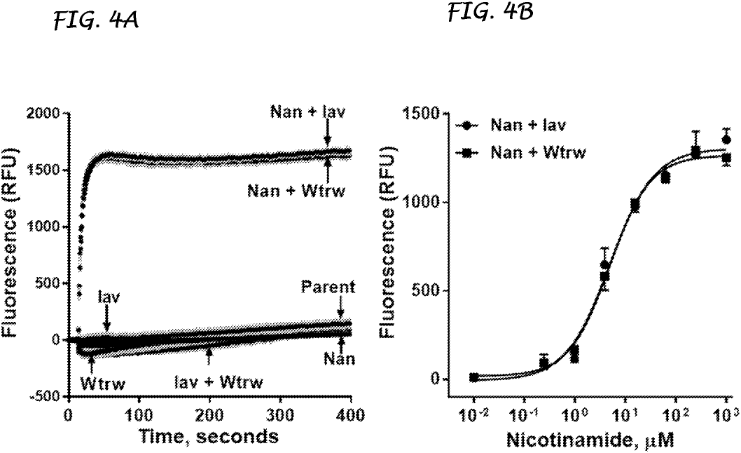

Nicotinamide, a form of vitamin B3, has been shown to activate both insect and nematode Nanchung and Inactive (Upadhyay et al., 2016). The stimulatory effects of nicotinamide required simultaneous expression of Nanchung and Inactive. To test whether nicotinamide can also activate Nanchung:Water witch complexes, Nanchung, Inactive and Water witch were expressed in CHO-K1 cells either individually, or in combinations. As shown on FIG. 4A, nicotinamide evoked Ca2+ response in cells co-expressing Nanchung and Water witch, and cells co-expressing Nanchung and Inactive, but not in the cells expressing these proteins individually. Furthemore, nicotinamide activated Nanchung:Inactive and Nancung: Water witch complexes with nearly identical potency and efficacy (FIG. 4B). As such, Nanchung:Water witch complexes can be activated by the same endogenous and exogenous modulators, which activate Nanchung:Inactive complexes.

### Example V: Co-expression of Nanchung With Water Witch Facilitates Binding Of Afidopyropen to Nanchung.

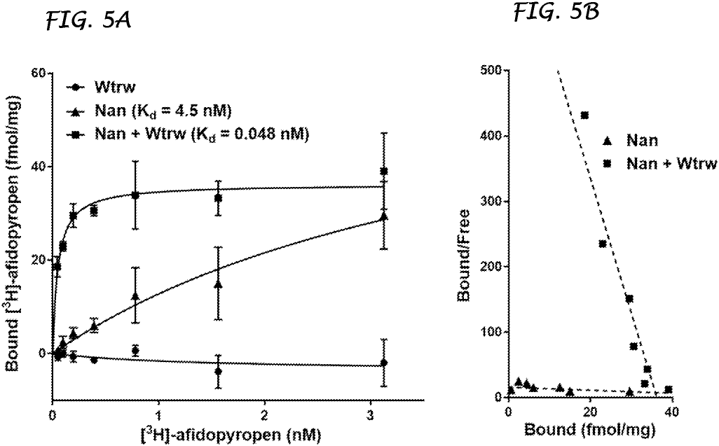

As depicted by FIG. 5A, specific binding of tritium-labeled afidopyropen to membranes prepared from CHO-K1 cells expressing Nanchung individually revealed the presence of a binding site with a relatively moderate affinity (K_{d} = 4.5 nM). Co-expression of Nanchung and Water witch increased binding affinity ca. 100-fold (Kd = 0.048 nM). Scatchard transformation of binding isotherms (FIG. 5B) further illustrates appearance of high affinity binding sites upon co-expression of Water witch with Nanchung. Binding of tritiated AP to membranes expressing Water witch alone was negligible. As such, analogous to previously reported increase of binding affinity upon co-expression of Nanchung and Inactive (Kandasamy et al., 2017), co-expression of Nanchung and Water witch dramatically facilitates binding of afidopyropen.

### Example VI: Pymetrozine and Nicotinamide Compete With Afidopyropen for Binding

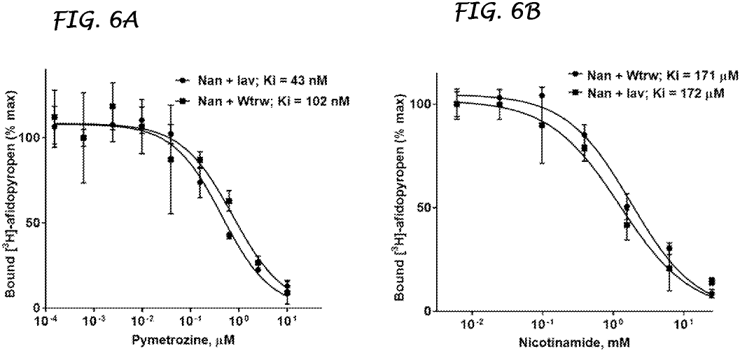

As depicted by FIG. 6, both pymetrozine (FIG. 6A) and nicotinamide (FIG. 6B) completely inhibited binding of tritiated afidopyropen to membranes prepared from cells co expressing Nanchung and Inactive, as well as cells co-expressing Nanchung and Water witch. These data indicate that afidopyropen, pymetrozine and nicotinamide have overlapping binding sites and suggest that agonist binding pockets in Nanchung-Inactive complex and Water witch-Inactive complex may be structurally similar.

### Example VII: Water Witch Forms a Functional Channel Activated by Plant-Derived Deterrents.

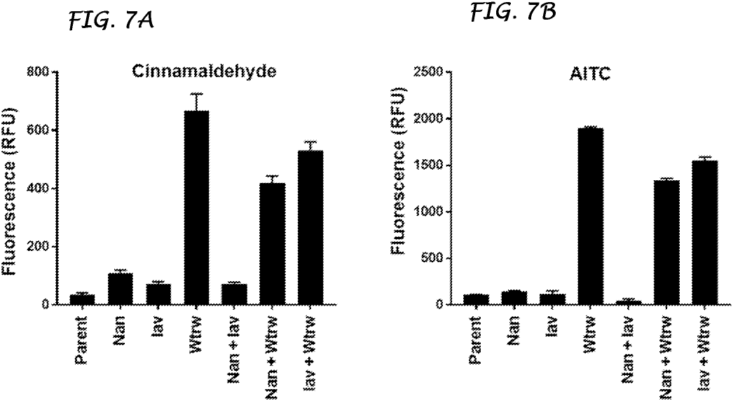

Biological activity of plant-derived deterrents and/or repellents allyl isothiocyanate and cinnamaldehyde has been attributed exclusively to activation of the insect TRPA1 channel. Based on amino acid sequence analysis, it has been determined that TRPA1 and Water witch proteins belong the different clades, TRPA1 clade and basal TRPA clade, respectively. Unlike TRPA1 clade, the members of the basal TRPA clade were predicted to be inefficient in detection of electrophilic compounds, which include allyl isothiocyanate and cinnamaldehyde (Kang et al., 2010). The inventors have found, however, that both individually expressed Water witch and Water witch co-expressed with Nanchung can be efficiently activated by allyl isothiocyanate and cinnamaldehyde (FIG. 7). Nanchung:Inactive complexes were not responsive to these compounds. This finding identifies Water witch protein as a new and alternative target for plant-derived deterrents and repellents, suggesting that assys which utilize heterologously expressed Water witch can be used for discovery of new compounds with deterrent and/or repellent properties.

### Example 8. Water Witch and Inactive Have Limited Similarity

Inactive and Water witch proteins belong to different subfamilies: TRPV and TRPA subfamilies respectively. Furthemore, amino acid alignment (FIG. 8) revealed that Drosopila melanogaster Inactive and Water witch proteins have only ca. 22% identity. Yet, the inventors demonstrated that Water witch protein is as capable of assembling with Nanchung protein, confer responses to agonists and facilitate afidopyropen binding as Inactive protein (Kandasamy et al., 2017; Nesterov et al., 2015). Given that Nanchung:Inactive and Nanchung:Water witch complexes interact with endogenous and exogenous agonists with nearly identical potencies, efficacies and affinities suggests that ligand binding pocket may be located entirely within Nanchung protein, whereas Water witch or Inactive act like chaperons to facilitate folding of Nanchung and/or change its conformation. This, in turn, raises the possibility that any other TRP channel capable of forming complex with Nanchung may act in similar fashion.

In summary, using the combination of functional and binding assays in the examples above, the inventors demonstrated the following: i) the insect proteins Nanchung and Water witch are capable of assembling into complex; ii) Nanchung-Water witch complex is a direct target of commercial insecticides afidopyropen and pymetrozine, as well as endogenous agonist nicotinamide; iii) afidopyropen, pymetrozine and nicotinamide have overlapping binding sites; iv) Nanchung protein forms the main binding interface for afidopyropen, whereas presence of Water witch protein increases binding affinity; v) Water witch protein is a direct target of plant-derived insecticides and repellents; vi) new methods were developed which can be used for identifying candidate compounds which act by directly targeting insect Water witch protein, or Nanchung-Water witch complexes and which may be used as insecticides or repellents.

### Materials and Methods

### Compounds

Pymetrozine was obtained from Sigma, flonicamid was purchased from ChemService Inc., (West Chester, PA) and TFNA-AM (4-trifluoronicotinamide) was obtained from ABCR Gmbh (Karlsruhe, Germany). Afidopyropen was synthesized at BASF. Tritium labeled Afidopyropen (19 Ci/mmol) was custom-made by RC TRITEC AG (Teufen, Germany).

### Heterologous Expression of Nanchung, Inactive and Water Witch

To produce expression constructs, complementary DNAs encoding for Drosophila melanogaster Water witch, (SEQ ID NO 63) and Inactive (NP_572353) were fused in-frame with FLAG tags and AcGFP fluorescent protein at carboxyl termini (FIG. 1). Complementary DNA encoding for Drosophila melanogaster Nanchung, (SEQ ID NO 7) was fused in-frame with HA tags and mCherry fluorescent protein at its carboxyl terminus (FIG. 1). The complimentary DNAs were codon-optimized for expression in mammalian cells.

To produce adenoviruses, complimentary DNAs encoding epitope-tagged TRP channels were subcloned into the adenovirus shuttle vector pENTCMV1-TetO (Welgen Inc., Worceter, MA). and ligated to a pAdREP plasmid (Welgen), which contains the remaining of the adenovirus genome. The pENTCMV1-TetO vector contains two Tet repressor binding sites within a modified CMV promoter, which repress transcription of the gene of interest in cells expressing the Tet repressor (FIG. 1). The recombination products were transformed into Escherichia coli cells, positive clones were selected, and the cosmid DNAs were isolated. The cosmid DNAs were transfected into HEK293-TetR cells, which produce the Tet repressor (Postle et al., 1984) preventing expression of TRP channels by the adenovirus packaging cells. Adenoviruses were purified from large-scale cultures and viral titers were calculated as viral particles/ml = Absorption at 260 nm x 1.1x 10¹⁴.

### Calcium Imaging

Hamster CHO-K1 cells (ATCC ® CCL-61TM) were infected with adenoviruses expressing TRP channels individually or in combinations. The cells were kept overnight at 37° C., followed by 3 days at 25° C. The medium was changed two days after seeding. Calcium mobilization was detected using fluorescent calcium probe fluo-4AM (Life Technologies) and measured by FLIPR-TETRA instrument (Molecular Devices, Sunnyvale, CA). The cells were loaded with 50 µl of Hank’s buffered salt solution (HBSS) containing 4 µM fluo-4AM, 5 mM probenecid, 20 mM CaCl₂, and 0.02% pluoronic for 2 hours at 25° C. The dye was then discarded, 50 µl of HBSS were added. The test compounds were dissolved in DMSO and added to the cells in 50 µl HBSS, yielding a final DMSO concentration of 0.2%. Fluorescence was monitored for 10 minutes at 1 second intervals at 470-495 nm/515-575 nm excitation/emission wavelengths. EC₅₀ values were calculated using GraphPad Prism (GraphPad Software).

### Membrane Preparation

The adenovirus-transduced CHO-K1 cells were seeded at 10 x 10⁶ cells per T175 flask (Greiner) and kept at 37° C. 18 hrs followed by incubation at 25° C. for 72 hrs. The media was changed at 24 hrs and 72 hrs post transduction. The cells were rinsed twice with PBS, and incubated in Versene solution (Life Technologies) for 10 min at room temperature. Detached cells were pelleted at 300 × g for 10 min and resuspended in 8 mL of ice-cold 20 mM Hepes (pH 7.3) containing 1% (wt/vol) protease inhibitors mixture (Halt; Thermo Fisher). After 10 min on ice, the cells were homogenized by sonication. The samples were centrifuged at 300 × g for 10 min, and supernatants were collected and centrifuged for 2 h at 50,000 × g. Membranes were resuspended in 0.5 mL of 50 mM HEPES, 100 mM NaCl, 1 mM CaCl₂, 5 mM MgCl₂, pH 7.4, and stored at -80° C. Protein content was determined using the Bradford Method.

### [³H]- Afidopyropen Binding Assay

Equilibrium binding assays were performed in the binding buffer (50 mM HEPES, 100mM NaCl, 1 mM CaCl₂, 5mM MgCl₂, pH 7.4). The reactions were performed in 96-well U-bottom polypropylene plates. 100 µL membranes resuspended in the binding buffer containing Tween-20 (0.0025% final concentration in the assay), were combined with either 200 µl (saturation binding experiments), or 800 µl (ligand displacement experiments) of binding buffer containing [³H]- afidopyropen, dissolved in ethanol, and, where indicated, test compounds, dissolved in DMSO. The final concentrations of ethanol and DMSO were kept at 0.2% and 0.25% respectfully in both saturation binding and competition experiments. Typically, 60-100 µg of membranes were used per data point. Followed by 3 hours of incubation at room temperature, assay mixes were filtered through Multiscreen HTS+ 96 Hi Flow FC filters (EMD Millipore), pre-wetted with 0.1% polyethyleneimine. Filters were washed three times with 200 µl of ice-cold binding buffer, scintillation cocktail was added and radioactivity was measured using Wallac 1450 Microbeta counter. Non-specific binding was measured in the presence of 500-fold excess of unlabeled afidopyropen. Kᵢ values were calculated by Cheng-Prusoff method (Ref)

### Western Blotting and Co-Immunoprecipitation

For Western blotting, adenovirus-transduced cells were seeded on 35 mm dishes. The cells were washed with phosphate-buffered saline and lysed in 300 µl of NuPAGE LDS sample buffer (Life Technologies) supplemented with a protease inhibitor cocktail (Sigma Aldrich) and TurboDNAse (Ambion). For co-immunoprecipitation, adenovirus-transduced cells were seeded on 90-mm dishes and lysed in 50 mM HEPES, 10% glycerol, 1% Triton-X-100, 100 mM NaCl, 1 mM EDTA, and 1 mM EGTA, containing 0.5 mM PMSF and the protease inhibitor cocktail (Sigma). FLAG-tagged Water witch protein was immunoprecipitated with anti-FLAG M2 affinity gel (Sigma). The samples were electrophoresed using the NuPaGe 4-12% Bis-Tris PreCast gel system (Life Technologies). Proteins were detected using anti-FLAG M2-peroxidase antibody (Sigma), or anti-HA-biotin antibody (Covance). Blots were developed with ECL reagent (Thermo Scientific).

Nanchung and Water witch sequence alignment was performed by Clustal W. Transmembrane domains in Inactive and Water witch were assigned by homology with cryo-electron structures of rat TRPV6 and human TRPA1 respectively using Molecular Operating Environment modeling software.

It is therefore intended that the foregoing detailed description be regarded as illustrative rather than limiting, and that it be understood that it is the following claims, including all equivalents, that are intended to define the spirit and scope of this invention.

1. A method for determining whether or not a candidate compound is a modulator of a complex consisting of an insect transient receptor potential channel Nanchung protein and an insect transient receptor potential channel Water witch protein, the method comprising:
(a) providing a first cell co-expressing the insect transient receptor potential channel Nanchung protein and the insect transient receptor potential channel Water witch protein;
(b) contacting the first cell with the candidate compound; and
(c) assaying for a modulation of the complex, wherein the modulation identifies the candidate compound as a modulator of the complex.

2. The method of claim 1, wherein the assaying step comprises at least one of the following steps:
(1) detecting calcium ion mobilization in the first cell in response to the candidate compound; or
(2) detecting a membrane potential in the first cell in response to the candidate compound.

3. The method of claim 1, wherein the assaying step comprises at least one of the following steps:
(1) comparing calcium ion mobilization in the first cell in the absence of the candidate compound with calcium ion mobilization in the first cell in the presence of the candidate compound; or
(2) comparing a membrane potential in the first cell in the absence of the candidate compound with a membrane potential in the first cell in the presence of the candidate compound.

4. The method of claim 1, wherein the assaying step comprises at least one of the following steps:
(1) comparing calcium ion mobilization in the first cell in the presence of the candidate compound with a calcium ion mobilization reference level indicative of no modulation of a complex consisting of an insect transient receptor potential channel Nanchung protein and an insect transient receptor potential channel Water witch protein; or
(2) comparing a membrane potential in the first cell in the presence of the candidate compound with a membrane potential reference level indicative of no modulation of a complex consisting of an insect transient receptor potential channel Nanchung protein and an insect transient receptor potential channel Water witch protein.

5. The method of claims 2-4, wherein the candidate compound modulates the calcium ion mobilization or the membrane potential in the first cell by at least 20% relative to the reference level.

6-24. (canceled)

25. A combination of expression vectors comprising:
(a) a first expression vector comprising a first nucleic acid molecule encoding a Nanchung protein, and
(b) a second expression vector comprising a second nucleic acid molecule encoding a Water witch protein.

26. (canceled)

27. The-combination of expression vectors of claim 25, wherein the first nucleic acid molecule comprises a nucleic acid sequence selected from SEQ ID NOS 1-27 and/or the second nucleic acid molecule comprises a nucleic acid sequence selected from SEQ ID NOS: 28-63.

28. (canceled)

29. An expression vector comprising:
(a) a first nucleic acid molecule encoding a Nanchung protein, and
(b) a second nucleic acid molecule encoding a Water witch protein.

30. (canceled)

31. The expression vector of claim 29 wherein the first nucleic acid molecule comprises a nucleic acid sequence selected from SEQ ID NOS: 1-27 and/or wherein the second nucleic acid molecule comprises a nucleic acid sequence selected from SEQ ID NOS: 28-63.

32. (canceled)

33. A cell comprising the expression vector of claims 29.

34. The cell of claim 33, wherein the Nanchung protein and the Water witch protein are co-expressed in the cell.

35-36. (canceled)

37. A method for determining whether a candidate compound binds directly to a complex consisting of an insect transient receptor potential channel Nanchung protein and an insect transient receptor potential channel Water witch protein, the method comprising:
(a) providing a complex consisting of an insect transient receptor potential channel Nanchung protein and an insect transient receptor potential channel Water witch protein;
(b) contacting the complex with a candidate compound; and
(c) determining a binding affinity of the candidate compound to the complex.

38-39. (canceled)

40. The method of claim 37, wherein the binding affinity is determined using one or more equilibrium binding constants, including K_{d} and/or Kᵢ.

41-63. (canceled)

64. A method for determining whether or not a candidate compound is a modulator of an insect transient receptor potential channel Water witch protein, comprising:
(a) providing a first cell expressing the insect transient receptor potential channel Water witch protein;
(b) contacting the first cell with the candidate compound; and
(c) assaying for a modulation of the insect transient receptor potential channel Water witch protein, wherein the modulation identifies the candidate compound as the modulator of the insect transient receptor potential channel Water witch protein.

65. The method of claim 64, wherein the assaying step comprises at least one of the following steps:
(1) detecting calcium ion mobilization in the first cell in response to the candidate compound; or
(2) detecting a membrane potential in the first cell in response to the candidate compound.

66. The method of claim 65, wherein the assaying step comprises at least one of the following steps:
(1) comparing calcium ion mobilization in the first cell in the absence of the candidate compound with calcium ion mobilization in the first cell in the presence of the candidate compound; or
(2) comparing a membrane potential in the first cell in the absence of the candidate compound with a membrane potential in the first cell in the presence of the candidate compound.

67. The method of claim 64, wherein the assaying step comprises at least one of the following steps:
(1) comparing calcium ion mobilization in the first cell in the presence of the candidate compound with a calcium ion mobilization reference level indicative of no modulation of the insect transient receptor potential channel Water witch protein; or
(2) comparing a membrane potential in the first cell in the presence of the candidate compound with a membrane potential reference level indicative of no modulation of an insect transient receptor potential channel Water witch protein.

68. The method of claim 65, wherein the candidate compound modulates the calcium ion mobilization or the membrane potential in the first cell by at least 20% relative to the reference level.

69. The method of claim 64, wherein the candidate compound is a modulator that inhibits the activity of the insect transient receptor potential channel Water witch protein.

70. The method of claim 64, wherein the candidate compound is a modulator that activates the insect transient receptor potential channel Water witch protein.

71-87. (canceled)

88. An expression vector comprising a nucleic acid molecule encoding a Water witch protein.

89. (canceled)

90. The expression vector of claim 88, wherein the nucleic acid molecule comprises a nucleic acid sequence selected from SEQ ID NOS: 28-63.

91. A cell comprising the expression vector of claim 88.

92-94. (canceled)
